(19) United States
(12) Patent Application Publication
(10) Pub. No.: US 2021/0283141 A1
(43) Pub. Date: Sep. 16, 2021

(54) PYRROLOBENZODIAZEPINE CONJUGATES

(71) Applicant: MEDIMMUNE LIMITED, Cambridge (GB)

(72) Inventors: Luke MASTERSON, Cambridge (GB); Balakumar VIJAYAKRISHNAN, Cambridge (GB); Ronald James CHRISTIE, Gaithersburg, MD (US)

(21) Appl. No.: 17/058,472

(22) PCT Filed: May 24, 2019
(86) PCT No.: PCT/EP2019/063423

§ 371 Date: Nov. 24, 2020

Related U.S. Application Data
(60) Provisional application No. 62/676,407, filed on May 25, 2018.

(51) Int. Cl. A61K 31/5517; A61K 47/68; A61K 47/55; A61K 47/54

(52) U.S. Cl. CPC A61K 31/5517; A61K 47/6849; A61K 47/545; A61K 47/55; A61K 47/6803

(57) ABSTRACT

A conjugate of formula I: wherein Ab is a modified antibody having at least one free conjugation site on each heavy chain and each of R^{LL1} and R^{LL2} comprise the group:

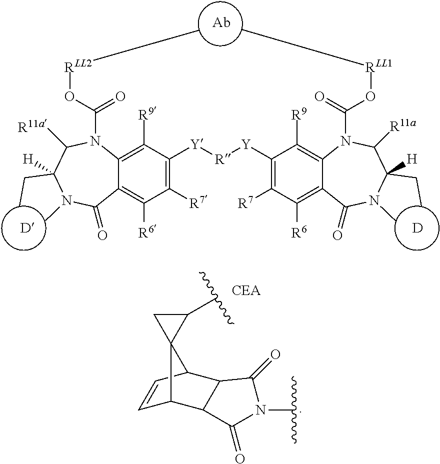

### CROSS-REFERENCE TO RELATED APPLICATIONS

This application is a national phase application under 35 U.S.C. § 371 of PCT International Application No. PCT/EP2019/063423, filed May 24, 2019, which claims priority to U.S. Provisional Application No. 62/676,407, filed May 25, 2018, each of which are hereby incorporated by reference in its entirety.

The present invention relates to conjugates comprising pyrrolobenzodiazepines and related dimers (PBDs).

### BACKGROUND TO THE INVENTION

Some pyrrolobenzodiazepines (PBDs) have the ability to recognise and bond to specific sequences of DNA; the preferred sequence is PuGPu. The first PBD antitumour antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and over 10 synthetic routes have been developed to a variety of analogues (Thurston, et al., Chem. Rev. 1994, 433-465 (1994)). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102)(Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)) and tomamycin (Arima, et al., J. Antibiotics, 25, 437-444 (1972)). PBDs are of the general structure:

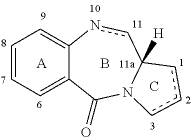

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as antitumour agents.

It has been previously disclosed that the biological activity of this molecules can be potentiated by joining two PBD units together through their C8/C′-hydroxyl functionalities via a flexible alkylene linker (Bose, D. S., et al., J. Am. Chem. Soc., 114, 4939-4941 (1992); Thurston, D. E., et al., J. Org. Chem., 61, 8141-8147 (1996)). The PBD dimers are thought to form sequence-selective DNA lesions such as the palindromic 5′-Pu-GATC-Py-3′ interstrand cross-link (Smellie, M., et al., Biochemistry, 42, 8232-8239 (2003); Martin, C., et al., Biochemistry, 44, 4135-4147) which is thought to be mainly responsible for their biological activity.

One example of a PBD dimer is SG2000 (SJG-136):

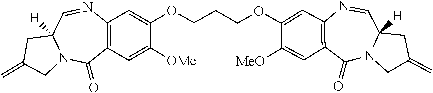

(Gregson, S., et al., J. Med. Chem., 44, 737-748 (2001); Alley, M. C., et al., Cancer Research, 64, 6700-6706 (2004); Hartley, J. A., et al., Cancer Research, 64, 6693-6699 (2004)) which has been involved in clinical trials as a standalone agent, for example, NCT02034227 investigating its use in treating Acute Myeloid Leukemia and Chronic Lymphocytic Leukemia (see: https://www.clinicaltrials.gov/ct2/show/NCT02034227).

Dimeric PBD compounds bearing C2 aryl substituents, such as SG2202 (ZC-207), are disclosed in WO 2005/085251:

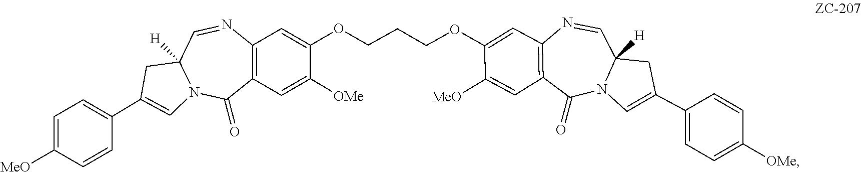 [SMILES: COc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

and in WO2006/111759, bisulphites of such PBD compounds, for example SG2285 (ZC-423):

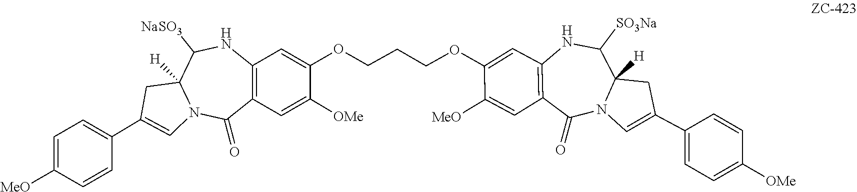

These compounds have been shown to be highly useful cytotoxic agents (Howard, P. W., et al., Bioorg. Med. Chem. (2009), doi: 10.1016/j.bmcl.2009.09.012).

WO 2007/085930 describes the preparation of dimer PBD compounds having linker groups for connection to a cell binding agent, such as an antibody. The linker is present in the bridge linking the monomer PBD units of the dimer.

Dimer PBD compounds having linker groups for connection to a cell binding agent, such as an antibody, are described in WO 2011/130598. The linker in these compounds is attached to one of the available N10 positions, and are generally cleaved by action of an enzyme on the linker group. If the non-bound N10 position is protected with a capping group, the capping groups exemplified have the same cleavage trigger as the linker to the antibody.

WO 2014/057074 describes two specific PBD dimer conjugates bound via the N10 position on one monomer, the other PBD monomer being in imine form. One of the drug-linkers disclosed is SG3249, Tesirine:

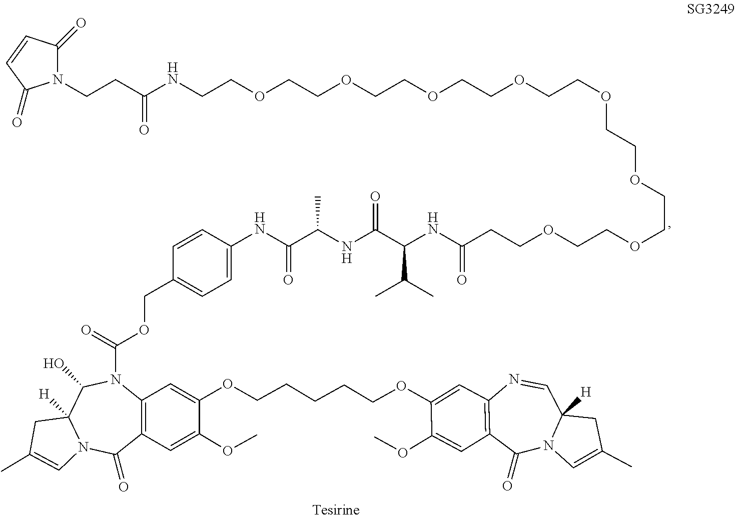 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)cc1)N=C[C@@H]1CC(C)=CN1C2=O]

which, when conjugated to anti-DLL3 rovalpituzumab, is know as rovalpituzumab-tesirine (Rova-T), currently under evaluation for the treatment of small cell lung cancer (Tiberghien, A. C., et al., ACS Med. Chem. Lett., 2016, 7 (11), 983-987, DOI: 10.1021/acsmedchemlett.6b00062). Further conjugates of this drug-linker with an engineered version of tratuzumab and a humanized antibody against human CD19 also began trials in early 2017 by ADC Therapeutics SA (Abstracts #51 and #52 in Proceedings of the American Association for Cancer Research, Volume 58, April 2017).

WO 2015/052322 describes a specific PBD dimer conjugate bound via the N10 position on one monomer, the other PBD monomer being in imine form. It also describes a specific PBD dimer conjugate bound via the N10 position on one monomer, the other PBD monomer having a capping group with the same cleavage trigger as the linker to the antibody:

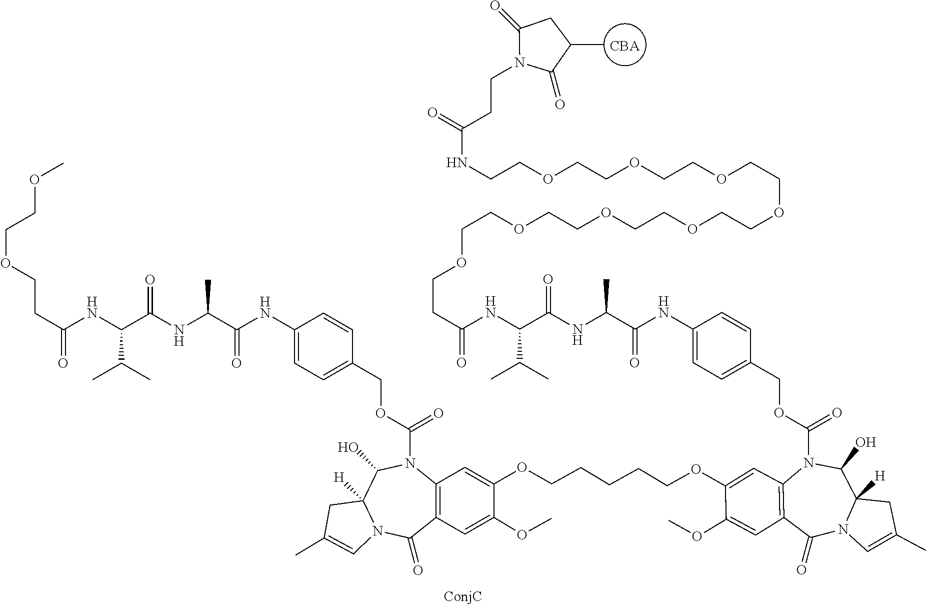 [SMILES: COCCOCCC(=O)N[C@H](C(=O)N[C@@H](C)C(=O)Nc1ccc(COC(=O)N2c3cc(OCCCCCOc4cc5c(cc4OC)C(=O)N4C=C(C)C[C@H]4[C@H](O)N5C(=O)OCc4ccc(NC(=O)[C@H](C)NC(=O)[C@@H](NC(=O)CCOCCOCCOCCOCCOCCOCCOCCOCCNC(=O)CCN5C(=O)CC(C)C5=O)C(C)C)cc4)c(OC)cc3C(=O)N3C=C(C)C[C@H]3[C@@H]2O)cc1)C(C)C]

### DISCLOSURE OF THE INVENTION

In one aspect the present invention provides PBD dimer conjugates wherein the PBDs are conjugated to antibodies that are modified so as to have at least one free conjugation site on each heavy chain, where the conjugation is via each N10 group of the PBD via a linker, and where the conjugation reaction is a Diels-reaction forming a cyclohexene ring.

The present inventors have found such conjugates to be surprisingly effective, despite the expectation that it was not possible to link a single PBD or related dimer to a single antibody by two linkers.

In another aspect the present invention provides PBD dimer conjugates wherein the PBDs are conjugated to antibodies via a N10 group of the PBD via a linker, and where the conjugation reaction is a Diels-reaction forming a cyclohexene ring.

A first aspect of the present invention provides a conjugate of formula I:

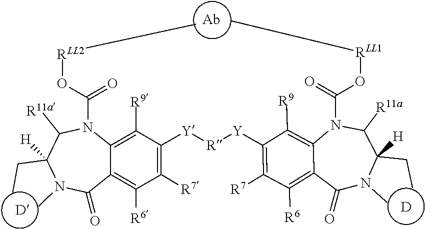

Wherein

Ab is a modified antibody having at least one free conjugation site on each heavy chain; D represents either group D1 or D2:

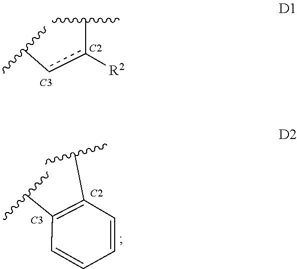

the dotted line indicates the optional presence of a double bond between C2 and C3: when there is a double bond present between C2 and C3, R² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id)

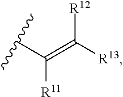

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(ie)

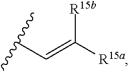

wherein one of R^{15a} and R^{15b} is H and the other is selected from: phenyl, which phenyl is optionally substituted by a group selected from halo, methyl, methoxy; pyridyl; and thiophenyl; and
(if)

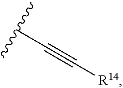

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2 and C3,

R² is selected from H, OH, F, diF and

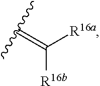

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

D′ represents either group D′1 or D′2:

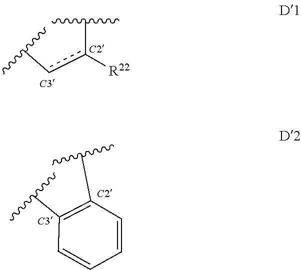

wherein the dotted line indicates the optional presence of a double bond between C2′ and C3′;

when there is a double bond present between C2′ and C3′, R¹² is selected from the group consisting of:

(iia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl;
(iid)

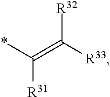

wherein each of R³¹, R³² and R³³ are independently selected from H, C₁₋₃ saturated alkyl, C₂₋₃ alkenyl, C₂₋₃ alkynyl and cyclopropyl, where the total number of carbon atoms in the R¹² group is no more than 5;

(iie)

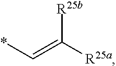

wherein one of R^{25a} and R^{25b} is H and the other is selected from: phenyl, which phenyl is optionally substituted by a group selected from halo, methyl, methoxy; pyridyl; and thiophenyl; and

(iif)

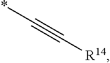

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2′ and C3′.

R¹² is selected from H, OH, F, diF and

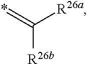

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester; R⁶ and R⁹ are independently selected from H, R, OH, OR, SH, SR, NH₂, NHR, NRR′, nitro, Me₃Sn and halo;

where R and R′ are independently selected from optionally substituted C₁₋₁₂ alkyl, C₃₋₂₀ heterocyclyl and C₅₋₂₀ aryl groups;

R⁷ is selected from H, R, OH, OR, SH, SR, NH₂, NHR, NRR′, nitro, Me₃Sn and halo;

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH;

R^{11a} is selected from OH, OR^{A}, where R^{A} is C₁₋₄ alkyl;

R^{6′}, R^{7′}, R^{9′} and R^{11a′} are selected from the same groups as R⁶, R⁷, R⁹ and R^{11a} respectively;

and

R^{LL1} and R^{LL2} are linkers connected to the antibody at different sites which are independently selected from:

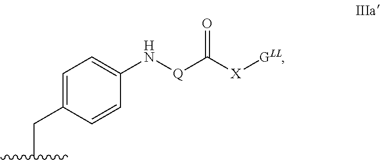

wherein

Q is:

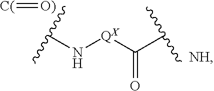

where Q^{X} is such that Q is an amino-acid residue, a dipeptide residue or a tripeptide residue;

X is:

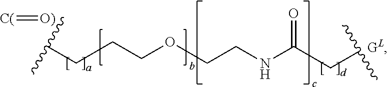

where a=0 to 5, b=0 to 16, c=0 or 1, d=0 to 5;

G^{LL} is a linker connected to the antibody comprising the group:

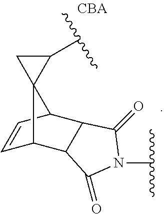 [SMILES: *C1CC12C1C=CC2C2C(=O)N(*)C(=O)C21]

It is thought that such ADCs which effectively have a drug antibody ratio (DAR) of 1 could offer significant advantages including reduced off-target toxicity and an enhanced therapeutic window by reducing the minimal effective dose requirement over ADCs consisting of heterogeneous mixtures with higher DARs.

A second aspect of the present invention comprises a conjugate of formula II:

Ab′-(D^{L})p (II),

where D^{L} is of formula (III)

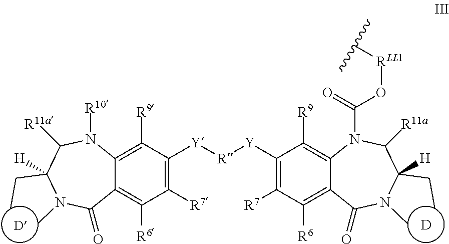

wherein D, R², R⁶, R⁷, R⁹, R^{11a}, Y, R″, Y′, D′, R^{6′}, R^{7′}, R^{9′}, R^{11a′} and R¹² (including the presence or absence of double bonds between C2 and C3 and C2′ and C3′ respectively) are as defined in the first aspect of the invention;

Ab′ is an antibody;

either:

(a) R^{10′} is H, and R^{11a′} is OH or OR^{A}, where R^{A} is C₁₋₄ alkyl;
(b) R^{10′} and R^{11a′} form a nitrogen-carbon double bond between the nitrogen and carbon atoms to which they are bound; or
(c) R^{10′} is H and R^{11a′} is SO_{z}M, where z is 2 or 3 and M is a monovalent pharmaceutically acceptable cation;

p is an integer of from 1 to 20.

Both the first and second aspects of the present invention have the group:

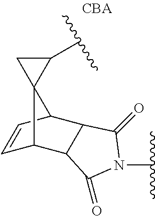 [SMILES: *C1CC12C1C=CC2C2C(=O)N(*)C(=O)C21]

in the linker between the PBD dimer and the antibody. Such a group may be formed by a Diels-Alder reaction between a maleimido group attached to the PBD dimer and a spirocyclopropyl-cyclopentadiene of formula:

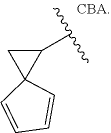 [SMILES: *C1CC12C=CC=C2]

Such a group can be incorporated into the antibody via the addition of a linker or by incorporating a non-natural amino acid into the polypeptide sequence.

Such a group provides a stable linkage between the antibody and the PBD dimer with the bridged cyclohexene ring.

A third aspect of the present invention provides the use of a conjugate of the first or second aspect of the invention in the manufacture of a medicament for treating a proliferative disease. The third aspect also provides a conjugate of the first or second aspect of the invention for use in the treatment of a proliferative disease. The third aspect also provides a method of treating a proliferative disease comprising administering a therapeutically effective amount of a conjugate of the first or second aspect of the invention to a patient in need thereof.

One of ordinary skill in the art is readily able to determine whether or not a candidate conjugate treats a proliferative condition for any particular cell type. For example, assays which may conveniently be used to assess the activity offered by a particular compound are described in the examples below.

### BRIEF DESCRIPTION OF FIGURES

FIG. 1.1. General design of spirocyclopentadiene crosslinkers (A) and spirocyclopentadiene NNAA (B) described in example 4.

FIG. 2.1. Shows intact deglycosylated mass spectra before (A) and after (B) reaction with CP2-NHS. Numbers below peaks in (B) indicate the number of CP2-linker groups introduced into the mAb. Estimation of CP2-linker introduction by peak intensities yields 3.29 CP2-linkers per mAb.

FIG. 3.1. Shows titers and cell viability of 12G3H11 K274CP2-NNAA mAb after expression in mammalian cells comprising mutant or wild type tRS.

FIG. 3.2. Shows deglycosylated mass spectra of 1C1 K274CP2-NNAA mAb.

FIG. 3.3. Shows deglycosylated mass spectrometry analysis of 1C1 S239CP2-NNAA mAb.

FIG. 3.4. Shows deglycosylated mass spectrometry analysis of 1C1 wild-type mAb.

FIG. 3.5. Shows SEC analysis of 1C1 K274CP2-NNAA mAb indicating that monomeric product was obtained.

FIG. 3.6. Shows SEC analysis of 1C1 S239CP2-NNAA mAb indicating that monomeric product was obtained.

FIG. 3.7. Shows analysis of 1C1-K274CP2-NNAA mAb and 1C1-S239CP2-NNAA mAb by SDS-PAGE.

### DEFINITIONS

### Pharmaceutically Acceptable Cations

Examples of pharmaceutically acceptable monovalent cations are discussed in Berge, et al., J. Pharm. Sci., 66, 1-19 (1977), which is incorporated herein by reference.

The pharmaceutically acceptable cation may be inorganic or organic.

Examples of pharmaceutically acceptable monovalent inorganic cations include, but are not limited to, alkali metal ions such as Na⁺ and K⁺. Examples of pharmaceutically acceptable divalent inorganic cations include, but are not limited to, alkaline earth cations such as Ca²⁺ and Mg²⁺. Examples of pharmaceutically acceptable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

### Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇). Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₁₂ Alkenyl: The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₁₂ alkynyl: The term “C₂₋₁₂ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);
unsaturated monocyclic hydrocarbon compounds:
   cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and
saturated polycyclic hydrocarbon compounds:
   norcarane (C₇), norpinane (C₇), norbornane (C₇).
   C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆); N₂O₁: oxadiazine (C₆);
O₁S₁: oxathiole (C₅) and oxathiane (thioxane) (C₆); and,
N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. The term “C₅₋₇ aryl”, as used herein, pertains to a monovalent moiety obtained by removing a hydrogen atom from an aromatic ring atom of an aromatic compound, which moiety has from 5 to 7 ring atoms and the term “C₅₋₁₀ aryl”, as used herein, pertains to a monovalent moiety obtained by removing a hydrogen atom from an aromatic ring atom of an aromatic compound, which moiety has from 5 to 10 ring atoms. Preferably, each ring has from 5 to 7 ring atoms.

In this context, the prefixes (e.g. C₃₋₂₀, C₅₋₇, C₅₋₆, C₅₋₁₀, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”.

Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to:
C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (Si), benzothiazole (N₁S₁), benzothiadiazole (N₂S);
C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ (with 2 fused rings) derived from benzodiazepine (N₂);
C₁₃ (with 3 fused rings) derived from carbazole (N₁), dibenzofuran (O₁), dibenzothiophene (S₁), carboline (N₂), perimidine (N₂), pyridoindole (N₂); and,
C₁₄ (with 3 fused rings) derived from acridine (N₁), xanthene (O₁), thioxanthene (Si), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇alkyl group.

Alkoxy: —OR, wherein R is an alkyl group, for example, a C₁₋₇ alkyl group. Examples of C₁₋₇ alkoxy groups include, but are not limited to, -OMe (methoxy), -OEt (ethoxy), -O(nPr) (n-propoxy), -O(iPr) (isopropoxy), -O(nBu) (n-butoxy), -O(sBu) (sec-butoxy), -O(iBu) (isobutoxy), and -O(tBu) (tert-butoxy).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): =0.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, =NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

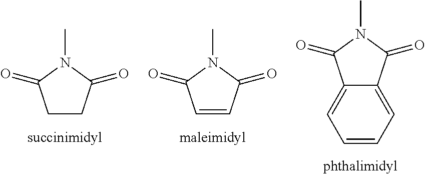

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

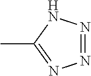

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇alkyl group. Examples of imino groups include, but are not limited to, ═NH, ═NMe, and =NEt.

Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group.

Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, where R is a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR)₂, where R is a phosphate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR)₂, where R is a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

### Alkylene

C₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH-₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

### Ligand Unit

The Ligand Units for use in the first aspect of the present invention are Cell Binding Agents, more specifically modified antibodies, or antigen binding fragments thereof, having at least one conjugation site on each heavy chain comprising a spirocyclopropyl-cyclopentadiene. Examples of partially modified antibodies suitable for use according to the present invention are disclosed in WO 2012/064733 (filed as PCT/US2011/059775), which is incorporated herein by reference. The antibodies disclosed would require further modification to provide the spirocyclopropyl-cyclopentadiene moieities, as described in the examples below.

The Ligand Units for use in the second aspect of the present invention are Cell Binding Agents, more specifically modified antibodies, or antigen binding fragments thereof, having at least one conjugation site comprising a spirocyclopropyl-cyclopentadiene.

In some embodiments of the first and second aspects, the PBDs are conjugated to the S239 position of the antibody.

### Antibodies

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include F(ab′)₂, and scFv fragments, and dimeric epitope-binding fragments of any of the above which immunospecifically bind to cancer cell antigens, viral antigens or microbial antigens, single-chain antibody molecules; and multispecific antibodies formed from antibody fragments.

Tumour-associate antigens and cognate antibodies for use in embodiments of the present invention are listed below, and are described in more detail on pages 14 to 86 of WO 2017/186894, which is incorporated herein.

(1) BMPR1B (bone morphogenetic protein receptor-type IB)

### (2) E16 (LAT1, SLC7A5)

(3) STEAP1 (six transmembrane epithelial antigen of prostate)

### (4) 0772P (CA125, MUC16)

(5) MPF (MPF, MSLN, SMR, megakaryocyte potentiating factor, mesothelin)
(6) NAPi3b (NAPI-3B, NPTIIb, SLC34A2, solute carrier family 34 (sodium phosphate), member 2, type II sodium-dependent phosphate transporter 3b)
(7) Sema 5b (FLJ10372, KIAA1445, Mm.42015, SEMA5B, SEMAG, Semaphorin 5b Hlog, 25 sema domain, seven thrombospondin repeats (type 1 and type 1-like), transmembrane domain (TM) and short cytoplasmic domain, (semaphorin) 5B)
(8) PSCA hlg (2700050C12Rik, C530008O 16Rik, RIKEN cDNA 2700050C12, RIKEN cDNA 2700050C12 gene)
(9) ETBR (Endothelin type B receptor)
(10) MSG783 (RNF124, hypothetical protein FLJ20315)
(11) STEAP2 (HGNC_8639, IPCA-1, PCANAP1, STAMP1, STEAP2, STMP, prostate cancer
associated gene 1, prostate cancer associated protein 1, six transmembrane epithelial antigen of prostate 2, six transmembrane prostate protein)
(12) TrpM4 (BR22450, FLJ20041, TRPM4, TRPM4B, transient receptor potential cation 5 channel, subfamily M, member 4)
(13) CRIPTO (CR, CR1, CRGF, CRIPTO, TDGF1, teratocarcinoma-derived growth factor)
(14) CD21 (CR2 (Complement receptor 2) or C3DR (C3d/Epstein Barr virus receptor) or Hs.73792)
(15) CD79b (CD79B, CD79β, IGb (immunoglobulin-associated beta), B29)
(16) FcRH2 (IFGP4, IRTA4, SPAP1A (SH2 domain containing phosphatase anchor protein 1a), SPAP1B, SPAP1C)

### (17) HER2 (ErbB2)

### (18) NCA (CEACAM6)

### (19) MDP (DPEP1)

### (20) IL20R-alpha (IL20Ra, ZCYTOR7)

### (21) Brevican (BCAN, BEHAB)

### (22) EphB2R (DRT, ERK, Hek5, EPHT3, Tyro5)

### (23) ASLG659 (B7h)

(24) PSCA (Prostate stem cell antigen precursor)

### (25) GEDA

(26) BAFF-R (B cell-activating factor receptor, BLyS receptor 3, BR3)
(27) CD22 (B-cell receptor CD22-B isoform, BL-CAM, Lyb-8, Lyb8, SIGLEC-2, FLJ22814)
(27a) CD22 (CD22 molecule)
(28) CD79a (CD79A, CD79alpha), immunoglobulin-associated alpha, a B cell-specific protein that covalently interacts with Ig beta (CD79B) and forms a complex on the surface with Ig M molecules, transduces a signal involved in B-cell differentiation), pl: 4.84, MW: 25028 TM: 2 [P] Gene Chromosome: 19q13.2).
(29) CXCR5 (Burkitt's lymphoma receptor 1, a G protein-coupled receptor that is activated by the CXCL13 chemokine, functions in lymphocyte migration and humoral defense, plays a
10 role in HIV-2 infection and perhaps development of AIDS, lymphoma, myeloma, and leukemia); 372 aa, pl: 8.54 MW: 41959 TM: 7 [P] Gene Chromosome: 11q23.3,
(30) HLA-DOB (Beta subunit of MHC class II molecule (Ia antigen) that binds peptides and 20 presents them to CD4+T lymphocytes); 273 aa, pl: 6.56, MW: 30820.TM: 1 [P] Gene Chromosome: 6p21.3)
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pl: 7.63, MW: 47206 TM: 1 [P] Gene Chromosome: 17p13.3).
(32) CD72 (B-cell differentiation antigen CD72, Lyb-2); 359 aa, pl: 8.66, MW: 40225, TM: 1 5 [P] Gene Chromosome: 9p13.3).
(33) LY64 (Lymphocyte antigen 64 (RP105), type I membrane protein of the leucine rich repeat (LRR) family, regulates B-cell activation and apoptosis, loss of function is associated
with increased disease activity in patients with systemic lupus erythematosis); 661 aa, pl: 6.20, MW: 74147 TM: 1 [P] Gene Chromosome: 5q12).
(34) FcRH1 (Fc receptor-like protein 1, a putative receptor for the immunoglobulin Fc domain
that contains C2 type Ig-like and ITAM domains, may have a role in B-lymphocyte 20 differentiation); 429 aa, pl: 5.28, MW: 46925 TM: 1 [P] Gene Chromosome: 1q21-1q22)
(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pl: 6.88, MW: 106468, TM: 1 [P] Gene Chromosome: 1q21)
(36) TENB2 (TMEFF2, tomoregulin, TPEF, HPP1, TR, putative transmembrane 35 proteoglycan, related to the EGF/heregulin family of growth factors and follistatin); 374 aa)
(37) PSMA-FOLH1 (Folate hydrolase (prostate-specific membrane antigen) 1)
(38) SST (Somatostatin Receptor; note that there are 5 subtypes)
(38.1) SSTR2 (Somatostatin receptor 2)
(38.2) SSTR5 (Somatostatin receptor 5)

### (38.3) SSTR1

### (38.4) SSTR3

### (38.5) SSTR4

AvB6—Both subunits (39+40)
(39) ITGAV (Integrin, alpha V)
(40) ITGB6 (Integrin, beta 6)
(41) CEACAM5 (Carcinoembryonic antigen-related cell adhesion molecule 5)
(42) MET (met proto-oncogene; hepatocyte growth factor receptor)
(43) MUC1 (Mucin 1, cell surface associated)
(44) CA9 (Carbonic anhydrase IX)
(45) EGFRvIII (Epidermal growth factor receptor (EGFR), transcript variant 3,
(46) CD33 (CD33 molecule)
(47) CD19 (CD19 molecule)
(48) IL2RA (Interleukin 2 receptor, alpha); NCBI Reference Sequence: NM_000417.2);
(49) AXL (AXL receptor tyrosine kinase)
(50) CD30-TNFRSF8 (Tumor necrosis factor receptor superfamily, member 8)
(51) BCMA (B-cell maturation antigen)-TNFRSF17 (Tumor necrosis factor receptor superfamily, member 17)

### (52) CT Ags-CTA (Cancer Testis Antigens)

(53) CD174 (Lewis Y)-FUT3 (fucosyltransferase 3 (galactoside 3(4)-L-fucosyltransferase, Lewis blood group)
(54) CLEC14A (C-type lectin domain family 14, member A; Genbank accession no. NM175060)
(55) GRP78-HSPA5 (heat shock 70 kDa protein 5 (glucose-regulated protein, 78 kDa)
(56) CD70 (CD70 molecule) L08096
(57) Stem Cell specific antigens. For example:
5T4 (see entry (63) below)
CD25 (see entry (48) above)
CD32
LGR5/GPR49
Prominin/CD133

### (58) ASG-5

(59) ENPP3 (Ectonucleotide pyrophosphatase/phosphodiesterase 3)
(60) PRR4 (Proline rich 4 (lacrimal))
(61) GCC-GUCY2C (guanylate cyclase 2C (heat stable enterotoxin receptor)
(62) Liv-1-SLC39A6 (Solute carrier family 39 (zinc transporter), member 6)
(63) 5T4, Trophoblast glycoprotein, TPBG-TPBG (trophoblast glycoprotein)
(64) CD56-NCMA1 (Neural cell adhesion molecule 1)
(65) CanAg (Tumor associated antigen CA242)

### (66) FOLR1 (Folate Receptor 1)

(67) GPNMB (Glycoprotein (transmembrane) nmb)
(68) TIM-1-HAVCR1 (Hepatitis A virus cellular receptor 1)
(69) RG-1/Prostate tumor target Mindin-Mindin/RG-1
(70) B7-H4-VTCN1 (V-set domain containing T cell activation inhibitor 1
(71) PTK7 (PTK7 protein tyrosine kinase 7)
(72) CD37 (CD37 molecule)
(73) CD138-SDC1 (syndecan 1)
(74) CD74 (CD74 molecule, major histocompatibility complex, class II invariant chain)

### (75) Claudins-CLs (Claudins)

(76) EGFR (Epidermal growth factor receptor)
(77) Her3 (ErbB3)-ERBB3 (v-erb-b2 erythroblastic leukemia viral oncogene homolog 3 (avian))
(78) RON-MST1R (macrophage stimulating 1 receptor (c-met-related tyrosine kinase))
(79) EPHA2 (EPH receptor A2)
(80) CD20-MS4A1 (membrane-spanning 4-domains, subfamily A, member 1)

### (81) Tenascin C-TNC (Tenascin C)

(82) FAP (Fibroblast activation protein, alpha)
(83) DKK-1 (Dickkopf 1 homolog (Xenopus laevis)
(84) CD52 (CD52 molecule)
(85) CS1-SLAMF7 (SLAM family member 7)

### (86) Endoglin-ENG (Endoglin)

### (87) Annexin A1-ANXA1 (Annexin A1)

(88) V-CAM (CD106)-VCAM1 (Vascular cell adhesion molecule 1)

### Methods of Treatment

The compounds of the present invention may be used in a method of therapy. Also provided is a method of treatment, comprising administering to a subject in need of treatment a therapeutically-effective amount of a conjugate of formula I. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g. drugs; surgery; and radiation therapy.

Pharmaceutical compositions according to the present invention, and for use in accordance with the present invention, may comprise, in addition to the active ingredient, i.e. a conjugate of formula I, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The Conjugates can be used to treat proliferative disease and autoimmune disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g., histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin.

Examples of autoimmune disease include the following: rheumatoid arthritis, autoimmune demyelinative diseases (e.g., multiple sclerosis, allergic encephalomyelitis), psoriatic arthritis, endocrine ophthalmopathy, uveoretinitis, systemic lupus erythematosus, myasthenia gravis, Graves' disease, glomerulonephritis, autoimmune hepatological disorder, inflammatory bowel disease (e.g., Crohn's disease), anaphylaxis, allergic reaction, Sjögren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), male and female autoimmune infertility, ankylosing spondolytis, ulcerative colitis, mixed connective tissue disease, polyarteritis nedosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the autoimmune disease is a disorder of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjögren's syndrome, Hashimoto's thyroiditis, Graves' disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes. In some embodiments, the autoimmunie disorder is a T cell-mediated immunological disorder.

In some embodiments, the amount of the Conjugate administered ranges from about 0.01 to about 10 mg/kg per dose. In some embodiments, the amount of the Conjugate administered ranges from about 0.01 to about 5 mg/kg per dose. In some embodiments, the amount of the Conjugate administered ranges from about 0.05 to about 5 mg/kg per dose. In some embodiments, the amount of the Conjugate administered ranges from about 0.1 to about 5 mg/kg per dose. In some embodiments, the amount of the Conjugate administered ranges from about 0.1 to about 4 mg/kg per dose. In some embodiments, the amount of the Conjugate administered ranges from about 0.05 to about 3 mg/kg per dose. In some embodiments, the amount of the Conjugate administered ranges from about 0.1 to about 3 mg/kg per dose. In some embodiments, the amount of the Conjugate administered ranges from about 0.1 to about 2 mg/kg per dose.

### Drug Loading

The drug loading (p) is the average number of PBD drugs per cell binding agent, e.g. antibody.

In the first aspect of the present invention, this is always 1. However, any composition may comprise antibodies where a PBD is conjugated and antibodies where a PBD is not conjugated. Thus for a composition, the drug loading (or DAR) may be less than 1, for example 0.75 and higher, 0.80 and higher, 0.85 and higher, 0.90 and higher or 0.95 or higher.

In the second of aspect, the drug loading is represented by p. The drug loading may range from 1 to 8 drugs (D) per cell binding agent, i.e. where 1, 2, 3, 4, 5, 6, 7, and 8 drug moieties are covalently attached to the cell binding agent. Compositions of conjugates include collections of cell binding agents, e.g. antibodies, conjugated with a range of drugs, from 1 to 8.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

In one embodiment, the average number of dimer pyrrolobenzodiazepine groups per cell binding agent is in the range 1 to 20. In some embodiments the range is selected from 1 to 8, 2 to 8, 2 to 6, 2 to 4, and 4 to 8.

### General Synthetic Routes

The synthesis of PBD compounds is extensively discussed in the following references, which discussions are incorporated herein by reference:

a) WO 00/12508 (pages 14 to 30);
b) WO 2005/023814 (pages 3 to 10);
c) WO 2004/043963 (pages 28 to 29); and
d) WO 2005/085251 (pages 30 to 39).

### Synthesis Route

Drug-linkers for use in making conjugates of the first aspect of the present invention of formula 1:

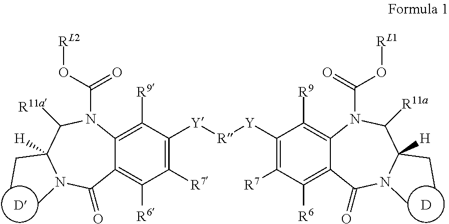

where R^{L1} and R^{L2} are groups that form R^{LL1} and R^{LL2} when conjugated to an antibody, can be synthesised from a compound of Formula 2:

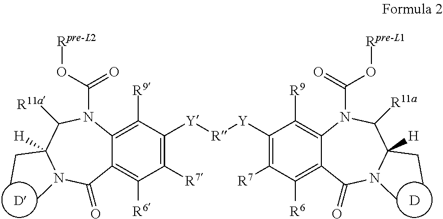

where R², R⁶, R⁷, R⁹, R^{11a}, R^{6′}, R^{7′}, R^{9′}, R^{11a′}, Y, Y′ and R″ are as defined for conjugates of the first aspect of the invention, R^{pre-L1} is a precursor of R^{L1} and R^{pre-L2} is a precursor of R^{L2}—this method is particularly applicable to compounds of formula I where R^{L1} and R^{L2} are of formula IIIa. For these compounds, R^{pre-L1} and R^{pre-L2} will typically be portions of R^{L1} and R^{L2}, such as a group of formula IIIa:

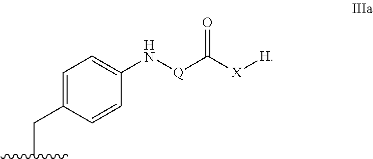

In such as case, the reaction involves adding the group G^{L} (the group that forms part of G^{LL} when conjugated).

The compounds of Formula 2 may be made by deprotecting compounds of Formula 3:

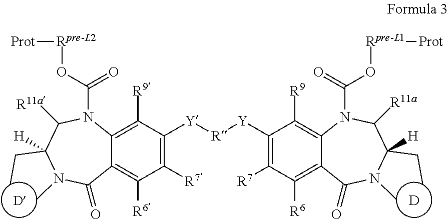

where R², R⁶, R⁷, R⁹, R^{11a}, R^{6′}, R^{7′}, R^{9′}, R^{11a′}, Y, Y′ and R″ are as defined for compounds of formula I, R^{pre-L1Prot} is a protected version of R^{pre-L1}, R^{pre-L2Prot} is a protected version of R^{pre-L2} and the Prot represents an appropriate carboxy/hydroxy protecting group.

Compounds of formula 3 may be made by ring-closure of compounds of Formula 4:

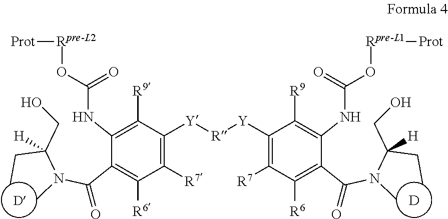

where the ring closure is carried out by oxidation, e.g. Swern.

Compounds of formula 4 can be synthesised from compounds of formula 5:

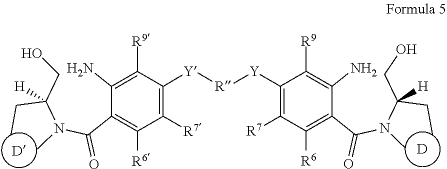

by addition of the two amino protecting groups. If the groups are different, step-wise addition can be achieved by simple protection of one amino group (e.g. by Fmoc), followed by installation of a desired protecting group at the other amino group. This can be followed by removal of the simple protecting group, and then installation of the other desired amino protecting group.

Compounds of Formula 5 can be synthesised by known methods, such as those disclosed in WO 2011/130598.

Drug linkers for use in forming the conjugates of the second aspect of the invention can be synthesised as described in the art, such as in WO2018/069490, WO2014/057074, WO2011/130598.

### Synthesis of Drug Conjugates

Antibodies can be conjugated to the Drug Linker compounds generally as described in the examples

### Further Preferences

The following preferences may apply to all aspects of the invention as described above, or may relate to a single aspect. The preferences may be combined together in any combination.

R^{6′}, R^{7′}, R^{9′}, R^{11a′} and Y′ are selected from the same groups as R⁶, R⁷, R⁹, R^{11a} and Y respectively. In some embodiments, R^{6′}, R^{7′}, R^{9′}, R^{11a′} and Y′ are the same as R⁶, R⁷, R⁹, R^{11a} and Y respectively.

In some embodiments, R¹² is the same as R².

### Dimer Link

In some embodiments, Y and Y′ are both O.

In some embodiments, R″ is a C₃₋₇ alkylene group with no substituents. In some of these embodiments, R″ is a C₃, C₅ or C₇ alkylene. In particular, R″ may be a C₃ or C₅ alkylene.

In other embodiments, R″ is a group of formula:

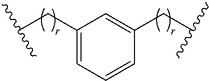

where r is 1 or 2.

R⁶ to R⁹

In some embodiments, R⁹ is H.

In some embodiments, R⁶ is selected from H, OH, OR, SH, NH₂, nitro and halo, and may be selected from H or halo. In some of these embodiments R⁶ is H.

In some embodiments, R⁷ is selected from H, OH, OR, SH, SR, NH₂, NHR, NRR′, and halo. In some of these embodiments R⁷ is selected from H, OH and OR, where R is selected from optionally substituted C₁₋₇ alkyl, C₃₋₁₀ heterocyclyl and C₅₋₁₀ aryl groups. R may be more preferably a C₁₋₄ alkyl group, which may or may not be substituted. A substituent of interest is a C₅₋₆ aryl group (e.g. phenyl). Particularly preferred substituents at the 7-positions are OMe and OCH₂Ph. Other substituents of particular interest are dimethylamino (i.e. —NMe₂); —(OC₂H₄)_{q}OMe, where q is from 0 to 2; nitrogen-containing C₆ heterocyclyls, including morpholino, piperidinyl and N-methyl-piperazinyl.

These embodiments and preferences apply to R^{9′}, R^{6′} and R^{7′} respectively.

### D and D′

In some embodiments, D and D′ are D1 and D′1 respectively.

In some embodiments, D and D′ are D2 and D′2 respectively.

### R²

When there is a double bond present between C2 and C3, R² is selected from:

(a) C₅₋₁₀ aryl group, optionally substituted by one or more substituents selected from the group comprising: halo, nitro, cyano, ether, C₁₋₇ alkyl, C₃₋₇ heterocyclyl and bis-oxy-C₁₋₃ alkylene;
(b) C₁₋₅ saturated aliphatic alkyl;
(c) C₃₋₆ saturated cycloalkyl;
(d)

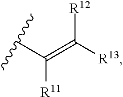

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(e)

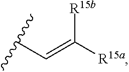

wherein one of R^{15a} and R^{15b} is H and the other is selected from: phenyl, which phenyl is optionally substituted by a group selected from halo methyl, methoxy; pyridyl; and thiophenyl; and
(f)

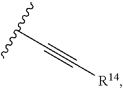

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo methyl, methoxy; pyridyl; and thiophenyl.

When R² is a C₅₋₁₀ aryl group, it may be a C₅₋₇ aryl group. A C₅₋₇ aryl group may be a phenyl group or a C₅₋₇ heteroaryl group, for example furanyl, thiophenyl and pyridyl. In some embodiments, R² is preferably phenyl. In other embodiments, R² is preferably thiophenyl, for example, thiophen-2-yl and thiophen-3-yl.

When R² is a C₅₋₁₀ aryl group, it may be a C₈₋₁₀ aryl, for example a quinolinyl or isoquinolinyl group. The quinolinyl or isoquinolinyl group may be bound to the PBD core through any available ring position. For example, the quinolinyl may be quinolin-2-yl, quinolin-3-yl, quinolin-4yl, quinolin-5-yl, quinolin-6-yl, quinolin-7-yl and quinolin-8-yl. Of these quinolin-3-yl and quinolin-6-yl may be preferred. The isoquinolinyl may be isoquinolin-1-yl, isoquinolin-3-yl, isoquinolin-4yl, isoquinolin-5-yl, isoquinolin-6-yl, isoquinolin-7-yl and isoquinolin-8-yl. Of these isoquinolin-3-yl and isoquinolin-6-yl may be preferred.

When R² is a C₅₋₁₀ aryl group, it may bear any number of substituent groups. It preferably bears from 1 to 3 substituent groups, with 1 and 2 being more preferred, and singly substituted groups being most preferred. The substituents may be any position.

Where R² is C₅₋₇ aryl group, a single substituent is preferably on a ring atom that is not adjacent the bond to the remainder of the compound, i.e. it is preferably p or y to the bond to the remainder of the compound. Therefore, where the C₅₋₇ aryl group is phenyl, the substituent is preferably in the meta- or para-positions, and more preferably is in the para-position.

Where R² is a C₆₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

R² Substituents, when R² is a C₅₋₁₀ Aryl Group

If a substituent on R² when R² is a C₅₋₁₀ aryl group is halo, it is preferably F or Cl, more preferably Cl.

If a substituent on R² when R² is a C₅₋₁₀ aryl group is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy). The alkoxy group may itself be further substituted, for example by an amino group (e.g. dimethylamino).

If a substituent on R² when R² is a C₅₋₁₀ aryl group is C₁₋₇ alkyl, it may preferably be a C₁₋₄ alkyl group (e.g. methyl, ethyl, propryl, butyl).

If a substituent on R² when R² is a C₅₋₁₀ aryl group is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups. If the C₆ nitrogen containing heterocyclyl group is piperazinyl, the said further substituent may be on the second nitrogen ring atom.

If a substituent on R² when R² is a C₅₋₁₀ aryl group is bis-oxy-C₁₋₃ alkylene, this is preferably bis-oxy-methylene or bis-oxy-ethylene.

If a substituent on R² when R² is a C₅₋₁₀ aryl group is ester, this is preferably methyl ester or ethyl ester.

Particularly preferred substituents when R² is a C₅₋₁₀ aryl group include methoxy, ethoxy, fluoro, chloro, cyano, bis-oxy-methylene, methyl-piperazinyl, morpholino and methylthiophenyl. Other particularly preferred substituents for R² are dimethylaminopropyloxy and carboxy.

Particularly preferred substituted R² groups when R² is a C₅₋₁₀ aryl group include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthiophenyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl. Another possible substituted R¹² group is 4-nitrophenyl. R¹² groups of particular interest include 4-(4-methylpiperazin-1-yl)phenyl and 3,4-bisoxymethylene-phenyl.

When R² is C₁₋₅ saturated aliphatic alkyl, it may be methyl, ethyl, propyl, butyl or pentyl. In some embodiments, it may be methyl, ethyl or propyl (n-pentyl or isopropyl). In some of these embodiments, it may be methyl. In other embodiments, it may be butyl or pentyl, which may be linear or branched.

When R² is C₃₋₆ saturated cycloalkyl, it may be cyclopropyl, cyclobutyl, cyclopentyl or cyclohexyl. In some embodiments, it may be cyclopropyl.

When R² is

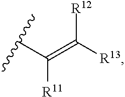

each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5. In some embodiments, the total number of carbon atoms in the R² group is no more than 4 or no more than 3.

In some embodiments, one of R¹¹, R¹² and R¹³ is H, with the other two groups being selected from H, C₁₋₃ saturated alkyl, C₂₋₃ alkenyl, C₂₋₃ alkynyl and cyclopropyl.

In other embodiments, two of R¹¹, R¹² and R¹³ are H, with the other group being selected from H, C₁₋₃ saturated alkyl, C₂₋₃ alkenyl, C₂₋₃ alkynyl and cyclopropyl.

In some embodiments, the groups that are not H are selected from methyl and ethyl. In some of these embodiments, the groups that re not H are methyl.

In some embodiments, R¹¹ is H.

In some embodiments, R¹² is H.

In some embodiments, R¹³ is H.

In some embodiments, R¹¹ and R¹² are H.

In some embodiments, R¹¹ and R¹³ are H.

In some embodiments, R¹² and R¹³ are H.

An R² group of particular interest is:

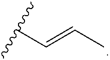 [SMILES: */C=C/C]

When R² is

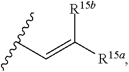

one of R^{15a} and R^{15b} is H and the other is selected from: phenyl, which phenyl is optionally substituted by a group selected from halo, methyl, methoxy; pyridyl; and thiophenyl. In some embodiments, the group which is not H is optionally substituted phenyl. If the phenyl optional substituent is halo, it is preferably fluoro. In some embodiment, the phenyl group is unsubstituted.

When R² is

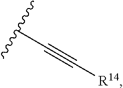

R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo methyl, methoxy; pyridyl; and thiophenyl. If the phenyl optional substituent is halo, it is preferably fluoro. In some embodiment, the phenyl group is unsubstituted.

In some embodiments, R¹⁴ is selected from H, methyl, ethyl, ethenyl and ethynyl. In some of these embodiments, R¹⁴ is selected from H and methyl.

When there is a single bond present between C2 and C3,

R² is H or

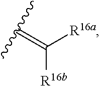

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester.

In some embodiments, R² is H.

In some embodiments, R² is

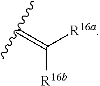

In some embodiments, it is preferred that R^{16a} and R^{16b} are both H.

In other embodiments, it is preferred that R^{16a} and R^{16b} are both methyl.

In further embodiments, it is preferred that one of R^{16a} and R^{16b} is H, and the other is selected from C₁₋₄ saturated alkyl, C₂₋₃ alkenyl, which alkyl and alkenyl groups are optionally substituted. In these further embodiment, it may be further preferred that the group which is not H is selected from methyl and ethyl.

### R²²

When there is a double bond present between C2′ and C3′, R²² is selected from:

(a) C₅₋₁₀ aryl group, optionally substituted by one or more substituents selected from the group comprising: halo, nitro, cyano, ether, C₁₋₇ alkyl, C₃₋₇ heterocyclyl and bis-oxy-C₁₋₃ alkylene;
(b) C₁₋₅ saturated aliphatic alkyl;
(c) C₃₋₆ saturated cycloalkyl;
(d)

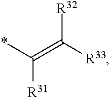

wherein each of R³¹, R³² and R³³ are independently selected from H, C₁₋₃ saturated alkyl, C₂₋₃ alkenyl, C₂₋₃ alkynyl and cyclopropyl, where the total number of carbon atoms in the R²² group is no more than 5;
(e)

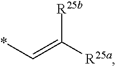

wherein one of R^{25a} and R^{25b} is H and the other is selected from: phenyl, which phenyl is optionally substituted by a group selected from halo methyl, methoxy; pyridyl; and thiophenyl; and
(f)

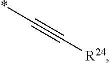

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo methyl, methoxy; pyridyl; and thiophenyl.

When R²² is a C₅₋₁₀ aryl group, it may be a C₅₋₇ aryl group. A C₅₋₇ aryl group may be a phenyl group or a C₅₋₇ heteroaryl group, for example furanyl, thiophenyl and pyridyl. In some embodiments, R²² is preferably phenyl. In other embodiments, R²² is preferably thiophenyl, for example, thiophen-2-yl and thiophen-3-yl.

When R²² is a C₅₋₁₀ aryl group, it may be a C₈₋₁₀ aryl, for example a quinolinyl or isoquinolinyl group. The quinolinyl or isoquinolinyl group may be bound to the PBD core through any available ring position. For example, the quinolinyl may be quinolin-2-yl, quinolin-3-yl, quinolin-4yl, quinolin-5-yl, quinolin-6-yl, quinolin-7-yl and quinolin-8-yl. Of these quinolin-3-yl and quinolin-6-yl may be preferred. The isoquinolinyl may be isoquinolin-1-yl, isoquinolin-3-yl, isoquinolin-4yl, isoquinolin-5-yl, isoquinolin-6-yl, isoquinolin-7-yl and isoquinolin-8-yl. Of these isoquinolin-3-yl and isoquinolin-6-yl may be preferred.

When R²² is a C₅₋₁₀ aryl group, it may bear any number of substituent groups. It preferably bears from 1 to 3 substituent groups, with 1 and 2 being more preferred, and singly substituted groups being most preferred. The substituents may be any position.

Where R²² is C₅₋₇ aryl group, a single substituent is preferably on a ring atom that is not adjacent the bond to the remainder of the compound, i.e. it is preferably p or y to the bond to the remainder of the compound. Therefore, where the C₅₋₇ aryl group is phenyl, the substituent is preferably in the meta- or para-positions, and more preferably is in the para-position.

Where R²² is a C₈₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

R²² Substituents, when R²² is a C₅₋₁₀ Aryl Group

If a substituent on R²² when R²² is a C₅₋₁₀ aryl group is halo, it is preferably F or Cl, more preferably Cl.

If a substituent on R²² when R²² is a C₅₋₁₀ aryl group is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy). The alkoxy group may itself be further substituted, for example by an amino group (e.g. dimethylamino).

If a substituent on R²² when R²² is a C₅₋₁₀ aryl group is C₁₋₇ alkyl, it may preferably be a C₁₋₄ alkyl group (e.g. methyl, ethyl, propryl, butyl).

If a substituent on R²² when R²² is a C₅₋₁₀ aryl group is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups. If the C₆ nitrogen containing heterocyclyl group is piperazinyl, the said further substituent may be on the second nitrogen ring atom.

If a substituent on R²² when R²² is a C₅₋₁₀ aryl group is bis-oxy-C₁₋₃ alkylene, this is preferably bis-oxy-methylene or bis-oxy-ethylene.

If a substituent on R²² when R²² is a C₅₋₁₀ aryl group is ester, this is preferably methyl ester or ethyl ester.

Particularly preferred substituents when R²² is a C₅₋₁₀ aryl group include methoxy, ethoxy, fluoro, chloro, cyano, bis-oxy-methylene, methyl-piperazinyl, morpholino and methylthiophenyl. Other particularly preferred substituents for R²² are dimethylaminopropyloxy and carboxy.

Particularly preferred substituted R²² groups when R²² is a C₅₋₁₀ aryl group include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthiophenyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl. Another possible substituted R²² group is 4-nitrophenyl. R²² groups of particular interest include 4-(4-methylpiperazin-1-yl)phenyl and 3,4-bisoxymethylene-phenyl.

When R²² is C₁₋₅ saturated aliphatic alkyl, it may be methyl, ethyl, propyl, butyl or pentyl. In some embodiments, it may be methyl, ethyl or propyl (n-pentyl or isopropyl). In some of these embodiments, it may be methyl. In other embodiments, it may be butyl or pentyl, which may be linear or branched.

When R²² is C₃₋₆ saturated cycloalkyl, it may be cyclopropyl, cyclobutyl, cyclopentyl or cyclohexyl. In some embodiments, it may be cyclopropyl.

When R²² is

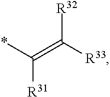

each of R³¹, R³² and R³³ are independently selected from H, C₁₋₃ saturated alkyl, C₂₋₃ alkenyl, C₂₋₃ alkynyl and cyclopropyl, where the total number of carbon atoms in the R²² group is no more than 5. In some embodiments, the total number of carbon atoms in the R²² group is no more than 4 or no more than 3.

In some embodiments, one of R³¹, R³² and R³³ is H, with the other two groups being selected from H, C₁₋₃ saturated alkyl, C₂₋₃ alkenyl, C₂₋₃ alkynyl and cyclopropyl.

In other embodiments, two of R³¹, R³² and R³³ are H, with the other group being selected from H, C₁₋₃ saturated alkyl, C₂₋₃ alkenyl, C₂₋₃ alkynyl and cyclopropyl.

In some embodiments, the groups that are not H are selected from methyl and ethyl. In some of these embodiments, the groups that re not H are methyl.

In some embodiments, R³¹ is H.

In some embodiments, R³² is H.

In some embodiments, R³³ is H.

In some embodiments, R³¹ and R³² are H.

In some embodiments, R³¹ and R³³ are H.

In some embodiments, R³² and R³³ are H.

An R²² group of particular interest is:

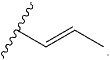 [SMILES: */C=C/C]

When R²² is

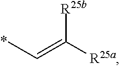

one of R^{25a} and R^{25b} is H and the other is selected from: phenyl, which phenyl is optionally substituted by a group selected from halo, methyl, methoxy; pyridyl; and thiophenyl. In some embodiments, the group which is not H is optionally substituted phenyl. If the phenyl optional substituent is halo, it is preferably fluoro. In some embodiment, the phenyl group is unsubstituted.

When R²² is

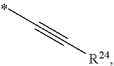

R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo methyl, methoxy; pyridyl; and thiophenyl. If the phenyl optional substituent is halo, it is preferably fluoro. In some embodiment, the phenyl group is unsubstituted.

In some embodiments, R²⁴ is selected from H, methyl, ethyl, ethenyl and ethynyl. In some of these embodiments, R²⁴ is selected from H and methyl.

When there is a single bond present between C2′ and C3′,

R²² is H or

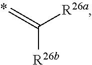

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester.

In some embodiments, R²² is H.

In some embodiments, R²² is

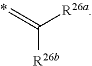

In some embodiments, it is preferred that R^{26a} and R^{26b} are both H.

In other embodiments, it is preferred that R^{26a} and R^{26b} are both methyl.

In further embodiments, it is preferred that one of R^{26a} and R^{26b} is H, and the other is selected from C₁₋₄ saturated alkyl, C₂₋₃ alkenyl, which alkyl and alkenyl groups are optionally substituted. In these further embodiment, it may be further preferred that the group which is not H is selected from methyl and ethyl.

### R¹¹

In some embodiments, R^{11a} is OH.

In some embodiments, R^{11a} is OR^{A}, where R^{A} is C₁₋₄ alkyl. In some of these embodiments, R^{A} is methyl.

In some embodiments of the first aspect conjugates of the present invention are of formula Ia, Ib or Ic:

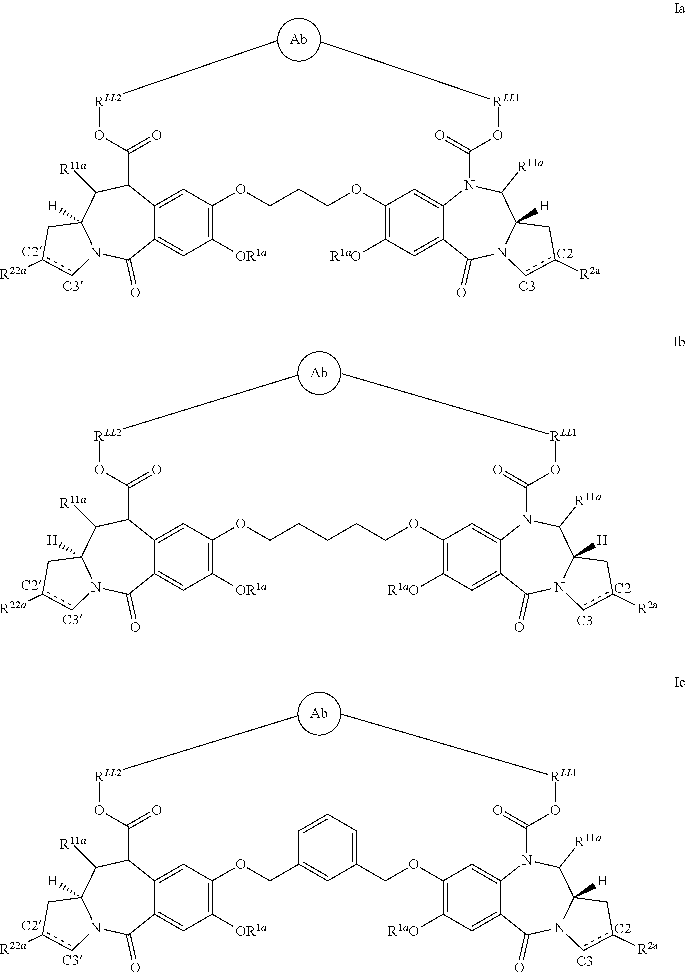

where R^{2a} and R^{22a} are the same and are selected from:

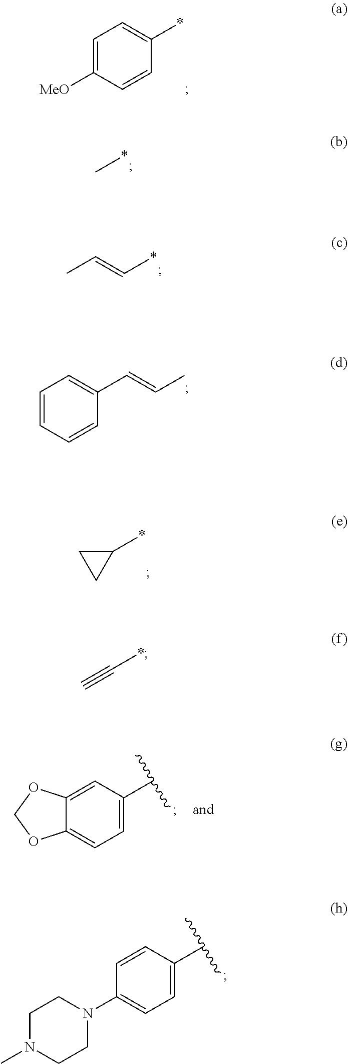

R^{1a} is selected from methyl and benzyl;

R^{LL1}, R^{LL2} and R^{11a} are as defined above.

In some embodiments of the second aspect conjugates of the present invention have D^{L} being of formula IIIa, IIIb or IIIc:

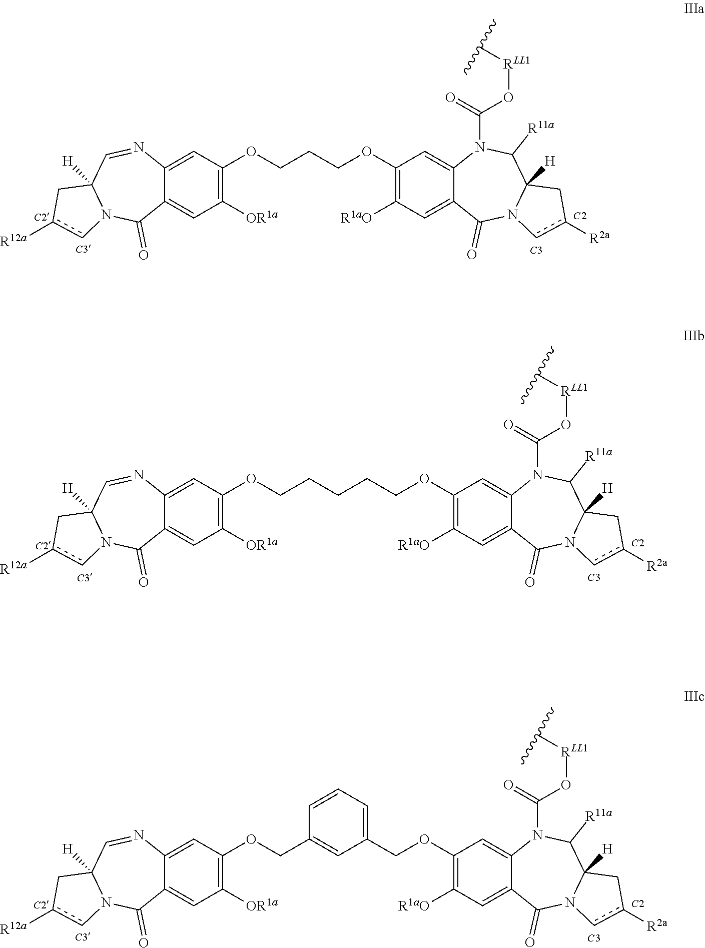

where R^{2a} and R^{22a} are the same and are selected from:

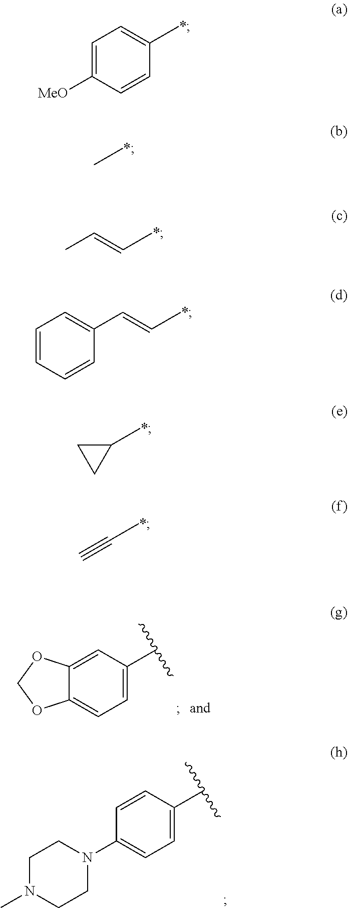

R^{1a} is selected from methyl and benzyl;

R^{LL1} and R^{LL2} are as defined above.

In some embodiments of the present invention both R² and R²² comprise no more than 3 carbon atoms.

Thus in these embodiments where there is a double bond present between C2 and C3, R² may be selected from:

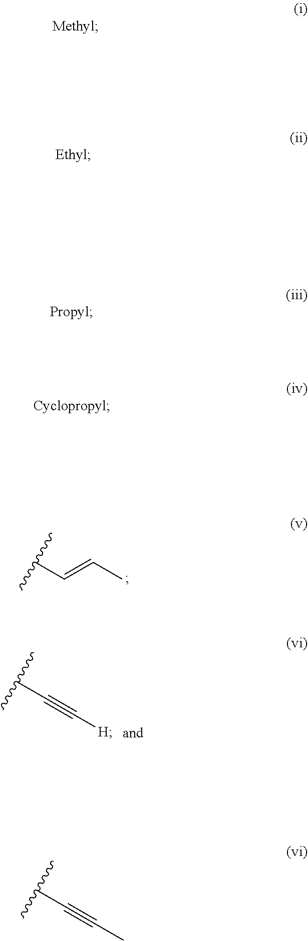

Thus in these embodiments where there is no double bond present between C2 and C3, R² may be selected from:

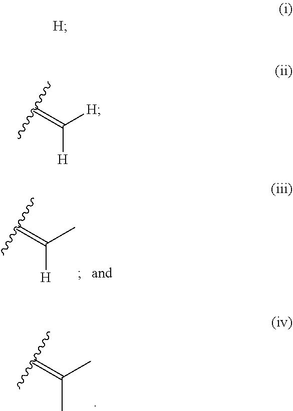

Thus in these embodiments where there is a double bond present between C2′ and C3′, R²² may be selected from:

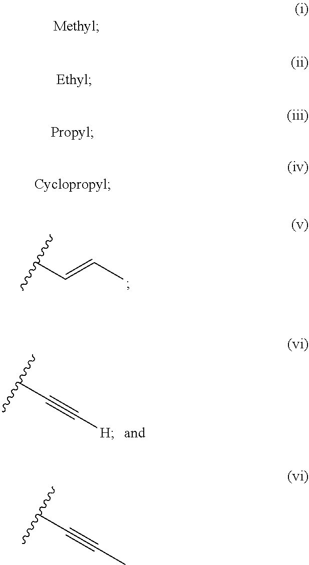

Thus in these embodiments where there is no double bond present between C2′ and C3′, R²² may be selected from:

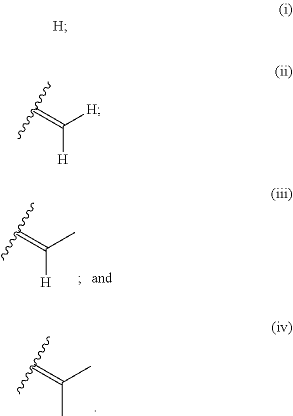

In some of these embodiments both R² and R²² comprise no more than 2 carbon atoms. Thus in these embodiments where there is a double bond present between C2 and C3, R² may be selected from:

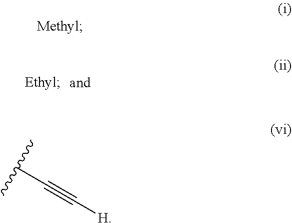

Thus in these embodiments where there is no double bond present between C2 and C3, R² may be selected from:

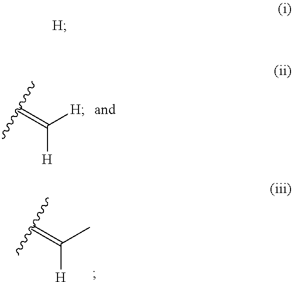

Thus in these embodiments where there is a double bond present between C2′ and C3′, R²² may be selected from:

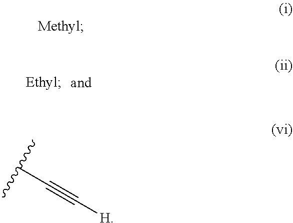

Thus in these embodiments where there is no double bond present between C2′ and C3′, R²² may be selected from:

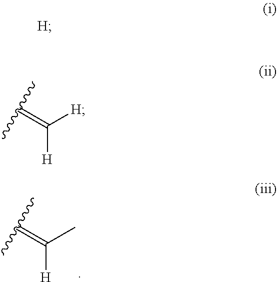

and

In further of these embodiments both R² and R²² comprise no more than 1 carbon atom.

Thus in these embodiments where there is a double bond present between C2 and C3, R² may be methyl. Thus in these embodiments where there is no double bond present between C2 and C3, R² may be selected from:

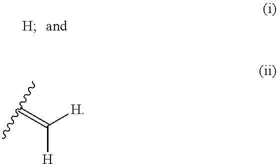

Thus in these embodiments where there is a double bond present between C2′ and C3′, R²² may be methyl. Thus in these embodiments where there is no double bond present between C2′ and C3′, R²² may be selected from:

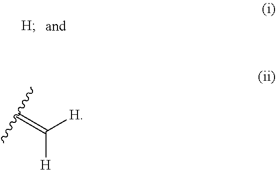

Without wishing to be bound by theory, where the substituent at the C2 position of the PBD dimers are small, the use of the glucuronide capping unit in these drug linkers is believed to be particularly advantageous, as it significantly increases the hydrophilicity of the drug linker, making the drug linkers easier to conjugate to a ligand unit.

These embodiments and preferences also apply to the second aspect of the invention, where appropriate.

R^{10′} and R^{11a′}

In one embodiment of the second aspect, R^{10′} is H, and R^{11a′} is OH or OR^{A}, where R^{A} is C₁₋₄ alkyl. In some of these embodiments, R^{A} is methyl.

In another embodiment of the second aspect, R^{10′} and R^{11a′} form a nitrogen-carbon double bond between the nitrogen and carbon atoms to which they are bound.

In another embodiment of the second aspect, R^{10′} is H and R^{11a′} is SO_{z}M, where z is 2 or 3 and M is a monovalent pharmaceutically acceptable cation.

### M

It is preferred that M is Na⁺.

### Linker (R^{LL})

### G^{LL}

G^{LL} may comprise a group selected from:

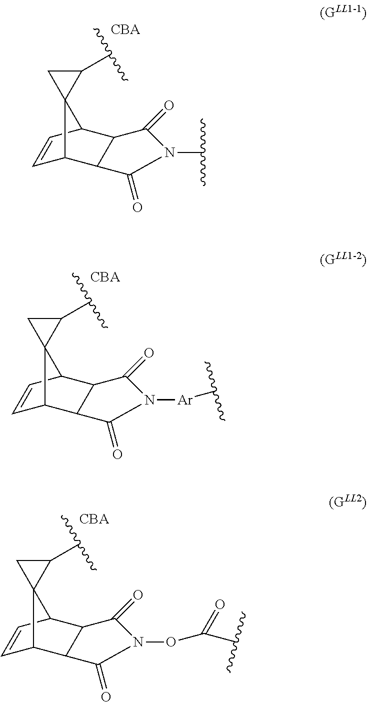

where Ar represents a C₅₋₆ arylene group, e.g. phenylene.

In some embodiments, G^{LL} comprises a group selected from G^{LL1-1} and G^{LL1-2}. In some of these embodiments, G^{LL} comprises G^{LL1-1}.

In some embodiments, the above groups (G^{LL1-1}, G^{LL1-2} and G^{LL2}) may be connected directly to X.

The above groups (G^{LL1-1}, G^{LL1-2} and G^{LL2}) may be connected to CBA via a group of formula IV:

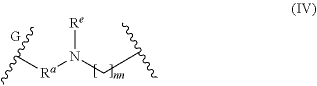

where G indicates where the group is connected to G^{LL1-1}, G^{LL1-2} and G^{LL2}

nn is from 1 to 4;

R^{a} represents a saturated or unsaturated (in particular saturated) branched or unbranched C₁₋₆ alkylene chain, wherein at least one carbon (for example 1, 2 or 3 carbons) is replaced by a heteroatom selected from O, N, S(O)₀₋₃, wherein said chain is optionally, substituted by one or more groups independently selected from oxo, halogen, amino; and

R^{e} represents H, saturated or unsaturated (in particular saturated) branched or unbranched C₁₋₈ alkylene chain, wherein one or more carbons are optionally replaced by —O— and the chain is optionally substituted by one or more halogen atoms (such as iodo), N₃ or —C₂₋₅ alkynyl.

In some embodiment R^{a} is —(CH₂)ₘC(O)—, —CH₂(CH₃)C(O)—, —(CH₂)ₘCH₂OC(O)—, —CHCHCH₂OC(O)—, or —OCH₂CH₂COc(O)— and m represents 0 or 1.

In some embodiments R^{e} represents H or —CH₂OCH₂CH₂N₃.

In some embodiments nn is 1. In other embodiments, nn is 2. In other embodiments, nn is 3. In other embodiments, nn is 4.

In some embodiments, the group is incorporated in the antibody by the use of an unnatural amino acid. Such an unnatural amino acid may be of formula AA:

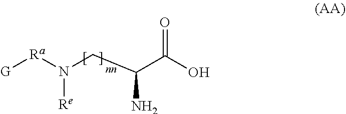

Where G is selected from a precursor of G^{LL1-1}, G^{LL1-2} and G^{LL2}.

In one of these embodiments, the unnatural amino acid is:

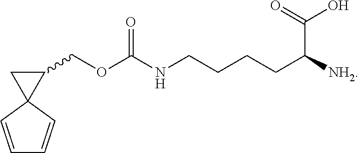 [SMILES: N[C@@H](CCCCNC(=O)OCC1CC12C=CC=C2)C(=O)O]

In another embodiment, the group is incorporated by conjugating a group of formula (BB) with the antibody. The site of conjugation might be a natural amino acid (such as a cysteine or a lysine) or a non-natural amino acid:

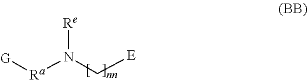

where E is a group —C(O)OR⁵⁵, R^{55′}, —NC(O)R⁶⁶, —C₂₋₅ alkylene, CH₂—O—NH₂ or halogen such as iodo;

R⁵⁵ represents C₁₋₆ alkyl, succinimide, C₆F₄H (tetrafluorohexyl), or H:

R^{55′} represents a sulfur bridging group, for example a dibromomaleimide, a dichloroacetone or a derivative of any one of the same,

R⁶⁶ represents:

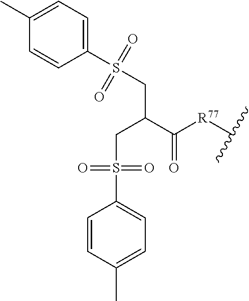

wherein

R⁷⁷ is C₁₋₆ alkylene optionally bearing one or more (such as one, two or three) groups selected from hydroxyl, sulfo, amino and —(OCH₂)_{V}C₂₋₆alkylene, and phenyl optionally bearing one or more (such as one, two or three) groups selected from hydroxyl, sulfo, amino and —(OCH₂)_{V}C₂₋₆alkylene,

v is an integer 1, 2, 3, 4 or 5

[symbol] represents where the fragment is connected to the rest of the molecule.

In one embodiment, the compound of formula BB is:

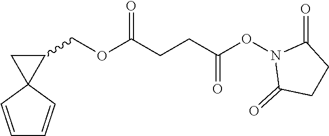 [SMILES: O=C(CCC(=O)ON1C(=O)CCC1=O)OCC1CC12C=CC=C2]

### X

X is:

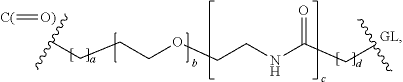

where a=0 to 5, b=0 to 16, c=0 or 1, d=0 to 5.

a may be 0, 1, 2, 3, 4 or 5. In some embodiments, a is 0 to 3. In some of these embodiments, a is 0 or 1. In further embodiments, a is 0.

b may be 0, 1, 2, 3, 4, 5, 6, 7, 8, 9, 10, 11, 12, 13, 14, 15 or 16. In some embodiments, b is 0 to 12. In some of these embodiments, b is 0 to 8, and may be 0, 2, 4 or 8.

c may be 0 or 1.

d may be 0, 1, 2, 3, 4 or 5. In some embodiments, d is 0 to 3. In some of these embodiments, d is 1 or 2. In further embodiments, d is 2.

In some embodiments of X, a is 0, c is 1 and d is 2, and b may be from 0 to 8. In some of these embodiments, b is 0, 4 or 8.

### Q^{X}

In one embodiment, Q^{X} is an amino acid residue. The amino acid may be an natural amino acids or a non-natural amino acid.

In one embodiment, Q^{X} is selected from: Phe, Lys, Val, Ala, Cit, Leu, lie, Arg, and Trp, where Cit is citrulline.

In one embodiment, Q^{X} comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment Q^{X} is selected from:

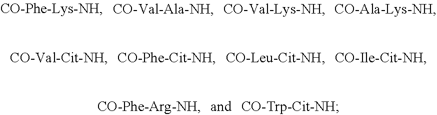

where Cit is citrulline.

Preferably, Q^{X} is selected from:

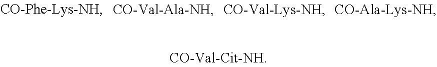

Most preferably, Q^{X} is selected from ^{CO}-Phe-Lys-^{NH}, ^{CO}-Val-Cit-^{NH} and ^{CO}-Val-Ala-^{NH}.

Other dipeptide combinations of interest include:

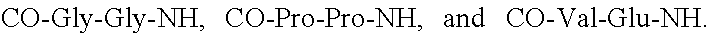

Other dipeptide combinations may be used, including those described by Dubowchik et al., Bioconjugate Chemistry, 2002, 13,855-869, which is incorporated herein by reference.

In some embodiments, Q^{X} is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin.

In one embodiment, the amino acid side chain is chemically protected, where appropriate. The side chain protecting group may be a group as discussed below. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

In one particular embodiment, the first aspect of the invention comprises a conjugate of formula Id:

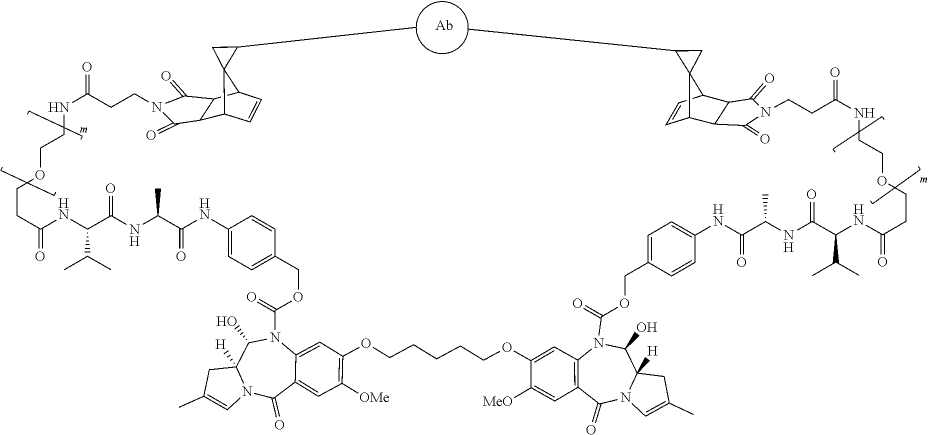

where m is an integer from 2 to 8.

In some embodiments, R^{LL1} and R^{LL2} are different.

In some embodiments, R^{LL1} and R^{LL2} are the same.

In particular, in embodiments where the linking groups are different, differences may only be in the G groups, such that the remainder of the linking groups are the same (so that the cleavage triggers are the same).

In one particular embodiment, the second aspect of the invention comprises a conjugate of where D^{L} is of formula IIId:

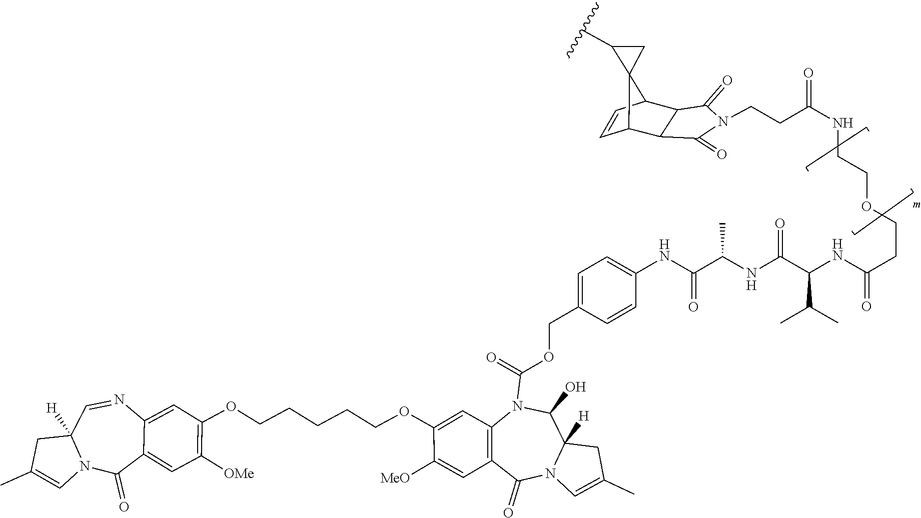

where m is an integer from 2 to 8.

In some embodiments of the present invention, the C11 substituent may be in the following stereochemical arrangement relative to neighbouring groups:

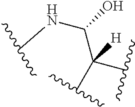

In other embodiments, the C11 substituent may be in the following stereochemical arrangement relative to neighbouring groups:

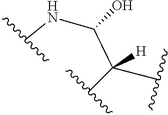

Compounds of particular interest include those of the examples.

### EXAMPLES

Flash chromatography was performed using silica gel under pressure. Fractions were checked for purity using thin-layer chromatography (TLC) using Merck Kieselgel 60 F254 silica gel, with fluorescent indicator on aluminium plates. Visualisation of TLC was achieved with UV light or iodine vapour unless otherwise stated. Extraction and chromatography solvents were bought and used without further purification from VWR U.K. All fine chemicals were purchased from Sigma-Aldrich unless otherwise stated. Pegylated reagents were obtained from Quanta biodesign US via Stratech UK or from Pierce Scientific via Thermo Fisher

¹H and ¹³C NMR spectra were obtained on a Bruker Avance® 400 spectrometer. Coupling constants are quoted in hertz (Hz). Chemical shifts are recorded in parts per million (ppm) downfield from tetramethylsilane. Spin multiplicities are described as s (singlet), bs (broad singlet), d (doublet), t (triplet), and m (multiplet).

The analytical LC/MS conditions (for reaction monitoring and purity determination) were as follows: Positive mode electrospray mass spectrometry was performed using a Shimadzu Nexera®/Prominence® LCMS-2020. Mobile phases used were solvent A (H₂O with 0.1% formic acid) and solvent B (CH₃CN with 0.1% formic acid). Gradient for routine 3-minute run: Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Gradient for 15-minute run: Initial composition 5% B held over 1.25 minutes, then increased from 5% B to 100% B over an 8.75 minute period. The composition was held for 2.5 minutes at 100% B, then returned to 5% B in 30 seconds and held there for 2 minutes. The total duration of the gradient run was 15.0 minutes. Flow rate was 0.8 mL/minute (for 3-minute run) and 0.5 mL/minute (for 15-minute run). Detection was at 254 nm. Columns: Waters Acquity UPLC® BEH Shield RP181.7 μm 2.1×50 mm at 50° C. fitted with Waters Acquity UPLC® BEH Shield RP18 VanGuard Pre-column, 130A, 1.7 μm, 2.1 mm×5 mm (routine 3-minute run); and Waters Acquity UPLC CSH C18, 1.7μ, 2.1×100 mm fitted with Waters Acquity UPLC® BEH Shield RP18 VanGuard Pre-column, 130A, 1.7 μm, 2.1 mm×5 mm (15 minute run).

The preparative HPLC conditions were as follows: Reverse-phase ultra-fast high-performance liquid chromatography (UFLC) was carried out on a Shimazdzu Prominence® machine using a Phenomenex® Gemini NX 5μ C18 column (at 50° C.) 150×21.2 mm. Eluents used were solvent A (H₂O with 0.05% formic acid) and solvent B (CH₃CN with 0.05% formic acid). All UFLC experiments were performed with gradient conditions: Initial composition 13% B, the composition was then increased to 100% B over a total of 17 minutes at a gradient suitable to effect the desired separation, then held for 1 minute at 100% B, then returned to 13% B in 0.1 minute and held there for 1.9 minutes. The total duration of the gradient run was 20.0 minutes. Flow rate was 20.0 mL/minute and detection was at 254 and 280 nm.

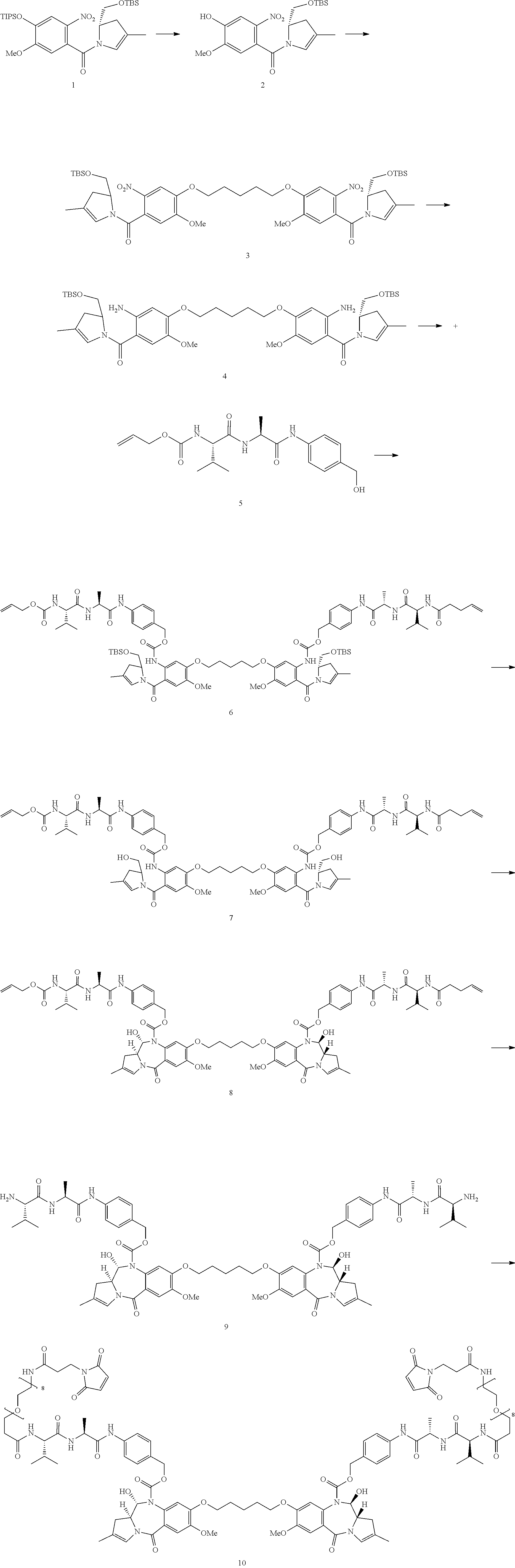

### (a) (S)-(2-(((tert-butyldimethylsilyl)oxy)methyl)-4-methyl-2,3-dihydro-1H-pyrrol-1-yl)(4-hydroxy-5-methoxy-2-nitrophenyl)methanone (2)

Lithium acetate dihydrate (3.52 g, 34.5 mmol, 1.0 eq.) was added to a stirred solution of TIPS ether (1) (19.96 g, 34.5 mmol, 1.0 eq.) in DMF/H₂O (300 mL/4 mL). The resultant red solution was stirred at room temperature for 3.5 h. The reaction mixture was diluted with EtOAc (600 mL) and washed with 1M citric acid solution (2×250 mL), H₂O (2×250 mL), saturated brine (300 mL) and dried (MgSO₄). The solvent was evaporated under reduced pressure to afford the product as a yellow solid (14.57 g, 100%). The product was used without further purification. Analytical Data: LC/MS, RT 1.74 min; MS (ES+) m/z (relative intensity) 423 ([M+H]⁺, 100); 445 ([M+Na])⁺, 75).

### (b) ((Pentane-1,5-diylbis(oxy))bis(5-methoxy-2-nitro-4,1-phenylene))bis(((S)-2-(((tert-butyldimethylsilyl)oxy)methyl)-4-methyl-2,3-dihydro-1H-pyrrol-1-yl)methanone) (3)

Potassium carbonate (5.03 g, 36.44 mmol, 1.1 eq.) was added to a stirred solution of phenol (2) (14 g, 33.13 mmol, 1.0 eq.) and 1,5 diiodopentane (21.46 g, 9.86 mL, 66.26 mmol, 2.0 eq.) in DMF (250 mL). The solution was heated at 70° C. for 3.5 h. The solution was poured into a mixture of ice/water (800 mL) and extracted with EtOAc (4×500 mL). The combined extracts were washed with H₂O (2×250 mL), saturated brine (400 mL), dried (MgSO₄) and evaporated under reduced pressure to give a brown oil. Purification by flash column chromatography [n-heptane/EtOAc 40% to 80% in 10% increments] gave the product as a yellow foam (12.7 g, 85%). Analytical Data: LC/MS, RT 2.16 min; MS (ES⁺) m/z (relative intensity) 913 ([M+H]⁺, 100); 935 ([M+Na])⁺, 100).

### (c) ((Pentane-1,5-diylbis(oxy))bis(2-amino-5-methoxy-4,1-phenylene))bis(((S)-2-(((tert-butyldimethylsilyl)oxy)methyl)-4-methyl-2,3-dihydro-1H-pyrrol-1-yl)methanone) (4)

Zinc dust (19.9 g, 304 mmol, 40 eq.) was treated with 1M HCl (100 mL) and stirred for 10 minutes at room temperature. The mixture was then sonicated for 10 minutes and the activated Zinc collected by vacuum filtration then washed with 1M HCl (50 mL), H₂O (to pH 6 to 7), MeOH and dried in vacuo on the filter pad. The activated zinc was added to a vigorously stirred solution of the bis nitro compound (3) (6.94 g, 7.6 mmol, 1.0 eq.) in EtOH/H₂O/EtOAc (60 mL/4 mL/60 mL) at room temperature. The reaction mixture was treated drop-wise with a solution of 5% v/v HCO₂H in MeOH (76 mL). A colour change from green to metallic grey and an exotherm to 42° C. were observed. Once the exotherm had subsided to 30° C. LC/MS indicated that the reaction was not complete. A further portion of 5% v/v HCO₂H in MeOH (20 mL) was added and a further exotherm was observed (34° C.) The reaction mixture was allowed to cool to room temperature at which point analysis by LC/MS revealed complete conversion to desired product. The mixture was filtered through Celite® and the pad washed with EtOAc. The filtrate was washed with saturated aqueous NaHCO₃ (2×300 mL), water (300 mL), saturated brine (300 mL), dried (MgSO₄), filtered and evaporated in vacuo to provide the bis-aniline as a yellow foam (6.22 g, 96%). The product was used without further purification. Analytical Data: LC/MS, RT 2.12 min; MS (ES⁺) m/z (relative intensity) 853 ([M+H]⁺, 15).

### (d) Bis(4-((S)-2-((S)-2-(((allyloxy)carbonyl)amino)-3-methylbutanamido)propanamido)benzyl) ((pentane-1,5-diylbis(oxy))bis(6-((S)-2-(((tert-butyldimethylsilyl)oxy)methyl)-4-methyl-2,3-dihydro-1H-pyrrole-1-carbonyl)-4-methoxy-3,1-phenylene))dicarbamate (6)

Triethylamine (0.171 g, 235 μL, 1.69 mmol, 4.4 eq.) was added via syringe to a stirred solution of bis aniline (4) (0.33 g, 0.38 mmol, 1.0 eq.) and triphosgene (0.082 g, 0.28 mmol, 0.72 eq.) in dry THF under an argon atmosphere. The resultant suspension was heated to 40° C. and after 5 min sampled in MeOH for LC/MS as the bis methyl carbamate (MS (ES+) m/z (relative intensity) 969 ([M+H]⁺, 80); 992 ([M+Na])⁺, 100). Dibutyltin dilaurate (0.024 g, 23 μL, 38 μmol, 0.1 eq.) then solid linker (5) (0.319 g, 0.85 mmol, 2.2 eq.) and trimethylamine (0.085 g, 118 μL, 0.85 mmol, 2.2 eq.) were added and the mixture heated at 40° C. with stirring under an argon atmosphere for 5 h. The reaction mixture was allowed to cool, filtered and the THF evaporated under reduced pressure. The residue was purified by flash column chromatography [CHCl₃/MeOH 0%, 1%, 1.5%, 2%, gradient elution] to give the product as a yellow foam (0.42 g, 66%). Analytical Data: LC/MS, RT 2.16 min; MS (ES⁺) m/z (relative intensity) 1660 ([M+H]⁺, 60); 1682 ([M+Na])⁺, 65).

### (e) Bis(4-((S)-2-((S)-2-(((allyloxy)carbonyl)amino)-3-methylbutanamido)propanamido)benzyl) ((pentane-1,5-diylbis(oxy))bis(6-((S)-2-(hydroxymethyl)-4-methyl-2,3-dihydro-1H-pyrrole-1-carbonyl)-4-methoxy-3,1-phenylene))dicarbamate (7)

p-Toluenesulfonic acid (0.296 g, 1.7 mmol, 2.2 eq.) was added to a stirred solution of bis-tert-butyldimethylsilyl ether (6) (1.26 g, 0.76 mmol, 1.0 eq.) in 10% v/v H₂O in THF. The solution was stirred at room temperature for 18 h. The reaction mixture was diluted with EtOAc (100 mL) and washed with saturated NaHCO₃ solution (2×100 mL), H₂O (100 mL), saturated brine (100 mL), dried (MgSO₄) and evaporated under reduced pressure. The residue was purified by flash column chromatography [CHCl₃/MeOH 0% to 5% in 1% increments] to give the product as a white foam (0.896 g, 92%). Analytical Data: LC/MS, RT 1.61 min; MS (ES⁺) m/z (relative intensity) 1432 ([M+H]⁺, 5); 1454 ([M+Na])⁺, 5).

### (f) Bis(4-((S)-2-((S)-2-(((allyloxy)carbonyl)amino)-3-methylbutanamido)propanamido)benzyl) 8,8′-(pentane-1,5-diylbis(oxy))(11S,11aS,11'S,11a'S)-bis(11-hydroxy-7-methoxy-2-methyl-5-oxo-11,11a-dihydro-1H-pyrrolo[2,1-c][1,4]benzodiazepine-10(5H)-carboxylate) (8)

Dess-Martin periodinane (0.24 g, 0.57 mmol, 2.0 eq.) was added to a stirred solution of bis-alcohol (7) in dry DCM (20 mL). The resultant white suspension was stirred at room temperature for 24 h. The reaction mixture was diluted with DCM (100 mL) and extracted with saturated NaHCO₃ solution (2×100 mL), water (100 mL), saturated brine (100 mL), dried (MgSO₄) and evaporated under reduced pressure. Purification by flash column chromatography [CHCl₃/MeOH 0% to 3% in 0.5% increments] gave the product as a white foam (0.28 g, 69%). Analytical Data: LC/MS, RT 1.58 min; MS (ES⁺) m/z (relative intensity) 1428 ([M+H]⁺, 20); 1450 ([M+Na])⁺, 30).

### (g) Bis(4-((S)-2-((S)-2-amino-3-methylbutanamido)propanamido)benzyl) 8,8′-(pentane-1,5-diylbis(oxy))(11S,11aS,11'S,11a'S)-bis(11-hydroxy-7-methoxy-2-methyl-5-oxo-11,11a-dihydro-1H-pyrrolo[2,1-c][1,4]benzodiazepine-10(5H)-carboxylate) (9)

Pd(PPh₃)₄ (8 mg, 7 μmol, 0.04 eq.) was added to a stirred solution of bis-alloc derivative (8) (0.25 g, 0.176 mmol 1.0 eq.) and pyrrolidine (31 mg, 36 μL 0.44 mmol, 2.5 eq.) in dry DCM (10 mL). The solution was stirred at room temperature for 2 h. The reaction mixture was partitioned between saturated NH₄Cl solution (50 mL) and DCM (50 mL). The DCM was separated and washed with saturated brine (100 mL), dried (MgSO₄) and evaporated under reduced pressure. The solid residue was triturated/sonicated with Et₂O (3×15 mL) and dried under vacuum to give the product as a white solid (0.207 g, 93%). The product was used without further purification. Analytical Data: LC/MS, RT 1.06 min; MS (ES⁺) m/z (relative intensity) 630 ([M+2H]⁺, 100).

### (h) Bis(4-((2S,5S)-37-(2,5-dioxo-2,5-dihydro-1H-pyrrol-1-yl)-5-isopropyl-2-methyl-4,7,35-trioxo-10,13,16,19,22,25,28,31-octaoxa-3,6,34-triazaheptatriacontanamido)benzyl) 8,8′-(pentane-1,5-diylbis(oxy))(11S,11aS,11'S,11a'S)-bis(11-hydroxy-7-methoxy-2-methyl-5-oxo-11,11a-dihydro-1H-pyrrolo[2,1-c][1,4]benzodiazepine-10(5H)-carboxylate) (10)

EDCl.HCl (56 mg, 0.29 mmol, 3 eq.) was added to a stirred solution of bis-amine (9) (0.123 g, 98 μmol, 1.0 eq.) and MaldPEG®OH (0.128 g, 0.22 mmol, 2.2 eq.) in CHCl₃ (15 mL). The reaction mixture was stirred at room temperature for 30 min then diluted with CHCl₃ (50 mL) washed with H₂O (100 mL), saturated brine (100 mL), dried (MgSO₄) and evaporated under reduced pressure. Purification by preparative HPLC followed by lyophilisation gave the product as a white foam (0.047 g, 20%). Analytical Data: LC/MS, RT 6.61 min; MS (ES⁺) m/z (relative intensity) 1205 ([M+2H]⁺, 55).

### Example 2

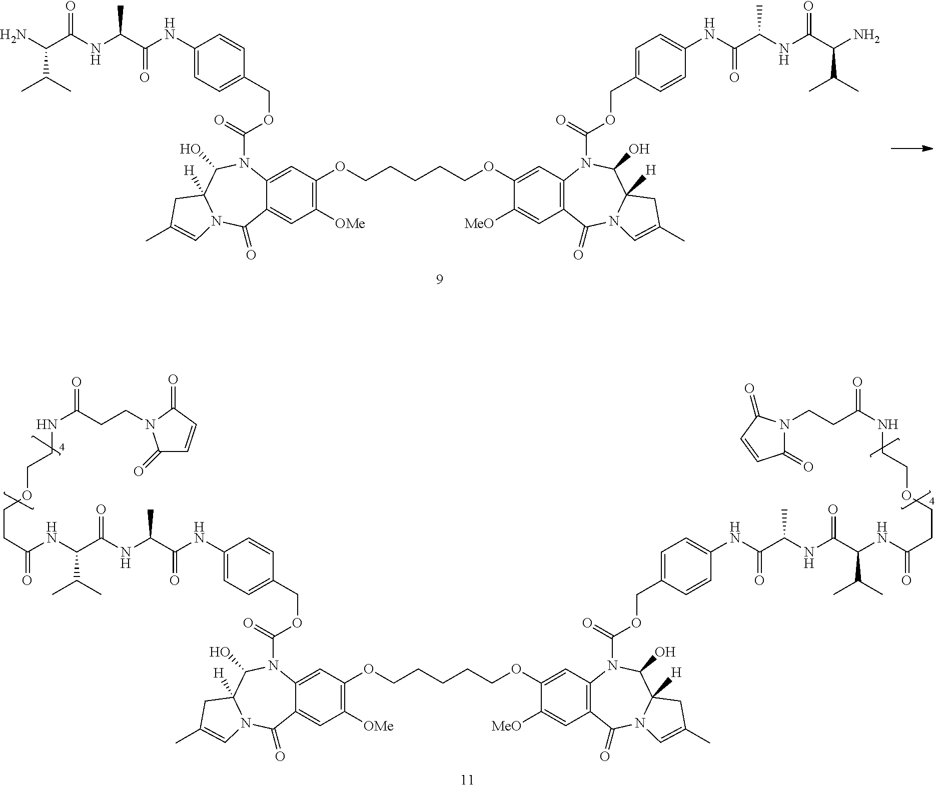

### Bis(4-((2S,5S)-25-(2,5-dioxo-2,5-dihydro-1H-pyrrol-1-yl)-5-isopropyl-2-methyl-4,7,23-trioxo-10,13,16,19-tetraoxa-3,6,22-triazapentacosanamido)benzyl) 8,8′-(pentane-1,5-diylbis(oxy))(11S,11aS,11'S,11a'S)-bis(11-hydroxy-7-methoxy-2-methyl-5-oxo-11,11a-dihydro-1H-pyrrolo[2,1-c][1,4]benzodiazepine-10(5H)-carboxylate) (11)

DIPEA (30 mg, 42 μL, 0.23 mmol, 3 eq.) was added to a stirred solution of bis-amine (9) (98 mg, 78 μmol, 1.0 eq.) and MalPEG₄OSu (88 mg, 0.17 mmol, 2.2 eq.) in CHCl₃ (10 mL). The reaction mixture was stirred at room temperature for 72 h then diluted with CHCl₃ (50 mL) washed with H₂O (100 mL), saturated brine (100 mL), dried (MgSO₄) and evaporated under reduced pressure. Purification by preparative HPLC followed by lyophilisation gave the product as a white foam (0.043 g, 25%). Analytical Data: LC/MS, RT 6.11 min; MS (ES⁺) m/z (relative intensity) 1028 ([M+2H]⁺, 80).

### Example 3

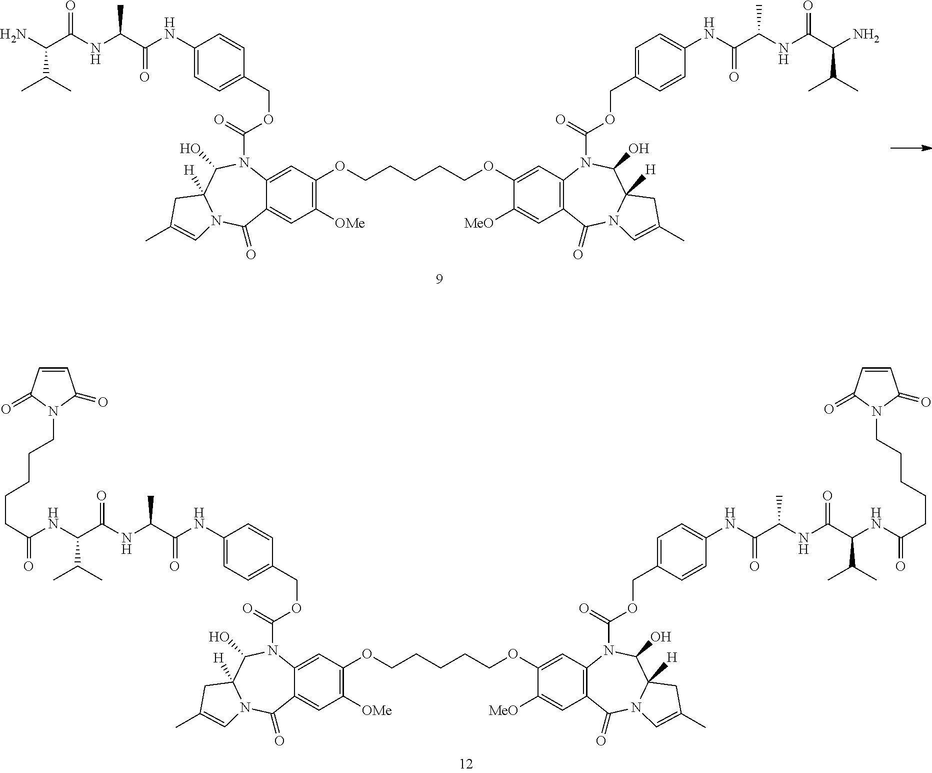 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](N)C(C)C)cc1)N(C(=O)OCc1ccc(NC(=O)[C@H](C)NC(=O)[C@@H](N)C(C)C)cc1)[C@@H](O)[C@@H]1CC(C)=CN1C2=O ; COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCCCCN3C(=O)C=CC3=O)C(C)C)cc1)N(C(=O)OCc1ccc(NC(=O)[C@H](C)NC(=O)[C@@H](NC(=O)CCCCCN3C(=O)C=CC3=O)C(C)C)cc1)[C@@H](O)[C@@H]1CC(C)=CN1C2=O]

### bis(4-((S)-2-((S)-2-(6-(2,5-dioxo-2,5-dihydro-1H-pyrrol-1-yl) hexanamido)-3-methylbutanamido)propanamido)benzyl) 8,8′-(pentane-1,5-diylbis(oxy))(11S,11aS,11'S,11a'S)-bis(11-hydroxy-7-methoxy-2-methyl-5-oxo-11,11a-dihydro-1H-pyrrolo[2,1-c][1,4]benzodiazepine-10(5H)-carboxylate) (12)

EDCl.HCl (50 mg, 0.26 mmol, 3 eq.) was added to a stirred solution of bis-amine (9) (0.109 g, 86.5 μmol, 1.0 eq.) and MCOSu (40 mg, 0.19 mmol, 2.2 eq.) in CHCl₃ (10 mL). The reaction mixture was stirred at room temperature for 30 min then diluted with CHCl₃ (50 mL) washed with H₂O (100 mL), saturated brine (100 mL), dried (MgSO₄) and evaporated under reduced pressure. Purification by preparative HPLC followed by lyophilisation gave the product as a white foam (0.045 g, 32%). Analytical Data: LC/MS, RT 6.82 min; MS (ES⁺) m/z (relative intensity) 1646 ([M+H]⁺, 20); 1667 ([M+Na])⁺, 30).

### Example 4

Spirocyclopentadiene-containint crosslinkers and non-natural amino acids (NNAAs) were prepared with the general structure shown below:

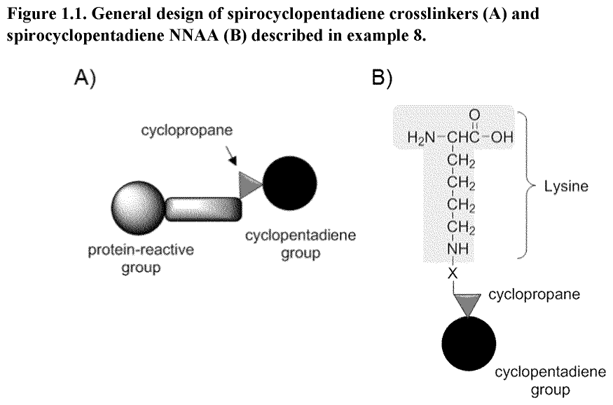

FIG. 1.1. General design of spirocyclopentadiene crosslinkers (A) and spirocyclopentadiene NNAA (B) described in example 4.

Synthesis of CP2-NNAA (16) began with the reaction of a commercially available NaCp solution with epichlorohydrin in a modified version of Carreira's reaction (Ledford, B. E.; Carreira, E. M., Total Synthesis of (+)-Trehazolin: Optically Active Spirocycloheptadienes as Useful Precursors for the Synthesis of Amino Cyclopentitols. Journal of the American Chemical Society 1995, 117, 11811-11812), Racemic epichlorohydrin was used, but 13 can be synthesized in 91% ee using enantiopure epichlorohydrin. The reaction of 13 with 4-nitrophenyl chloroformate produced activated carbamate 14. Reacting 14 with Fmoc-Lys-OH produces the Fmoc-protected 15, which could be deprotected using piperidine to obtain NNAA 16. None of the intermediates in its synthesis show dimerization or decomposition when stored at −20° C.

The synthesis of CP2-functionalized NHS-ester 18 began with the reaction of 13 with succinic anhydride to produce acid 17. The acid 17 was reacted with EDC-HCl and N-hydroxysuccinimide to yield NHS ester 18. Compound 18 doesn't appear to dimerize when stored for several days at room temperature.

Materials and Methods: Unless stated otherwise, reactions were conducted under an atmosphere of N₂ using reagent grade solvents. DCM, and toluene were stored over 3 Å molecular sieves. THF was passed over a column of activated alumina. All commercially obtained reagents were used as received. Thin-layer chromatography (TLC) was conducted with E. Merck silica gel 60 F254 pre-coated plates (0.25 mm) and visualized by exposure to UV light (254 nm) or stained with p-anisaldehyde, ninhydrin, or potassium permanganate. Flash column chromatography was performed using normal phase silica gel (60 Å, 0.040-0.063 mm, Geduran). ¹H NMR spectra were recorded on Varian spectrometers (400, 500, or 600 MHz) and are reported relative to deuterated solvent signals. Data for ¹H NMR spectra are reported as follows: chemical shift (6 ppm), multiplicity, coupling constant (Hz) and integration. ¹³C NMR spectra were recorded on Varian Spectrometers (100, 125, or 150 MHz). Data for ¹³C NMR spectra are reported in terms of chemical shift (6 ppm). Mass spectra were obtained from the UC Santa Barbara Mass Spectrometry Facility on a (Waters Corp.) GCT Premier high resolution Time-of-flight mass spectrometer with a field desorption (FD) source.

### (i) Synthesis of CP2-NNAA (16)

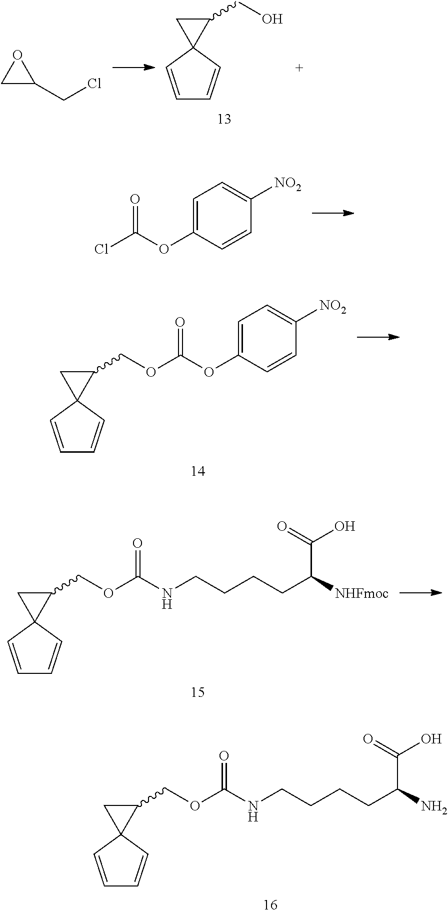

### (a) Spiro[2.4]hepta-4,6-dien-1-ylmethanol (13)

Sodium cyclopentadienide (2 M solution in THF, 10 mL, 20 mmol, 4 eq) was added to THF (40 mL) and cooled to 0° C. Epichlorohydrin (0.39 mL, 5.0 mmol, 1 eq) was added dropwise and the reaction was stirred at 0° C. for 1.5 hr then a further 2 hr at rt. The reaction was quenched with H₂O (40 mL) then transferred to a seperatory funnel. A saturated solution of NaHCO₃ in H₂O (40 mL) and ether (40 mL) were added and the layers separated. The organic layer was washed with brine (40 mL), dried over MgSO₄, filtered, and then the solvent removed. The residue was subjected to flash column chromatography (Hexane:EtOAc, 2:1) to yield 13 (0.48 g, 78%) as a brown oil.

Rf (Hexane:EtOAc, 2:1): 0.22; ¹H NMR (500 MHz, CDCl₃) δ 6.64 (td, J=1.6, 5.1 Hz, 1H), 6.51 (td, J=1.7, 5.1 Hz, 1H), 6.27 (tdd, J=1.0, 2.1, 5.2 Hz, 1H), 6.12 (td, J=1.7, 5.1 Hz, 1H), 4.08-3.88 (m, 1H), 3.59 (dd, J=8.8, 11.7 Hz, 1H), 2.48-2.40 (m, 1H), 1.87 (dd, J=4.3, 8.7 Hz, 1H), 1.69 (dd, J=4.4, 7.0 Hz, 1H), 1.57 (br. s., 1H) ppm; ¹³C NMR (125 MHz, CDCl₃) δ 139.4, 133.9, 131.7, 128.6, 64.9, 41.9, 30.0, 17.6 ppm.

### (b) 4-Nitrophenyl spiro[2.4]hepta-4,6-dien-1-ylmethyl carbonate (14)

13 (2.80 g, 22.9 mmol, 1 eq) was added to DCM (100 mL) and cooled to 0° C. Pyridine (4.61 mL, 57.3 mmol, 2.5 eq) was added followed by 4-nitrophenyl chloroformate (5.08 g, 25.2 mmol, 1.1 eq). The reaction was stirred at 0° C. until consumption of the starting material (TLC, 30 min). The reaction was poured into a separatory funnel and washed with a saturated solution of NH₄Cl in H₂O (100 mL). The aqueous layer was extracted with DCM (50 mL). The organic layers were combined, washed with brine (50 mL), dried over Na₂SO₄, filtered, and the solvent removed. The residue was subjected to flash column chromatography (Hexane:EtOAc, 6:1 to 4:1) to yield 14 (5.17 g, 79%) as an amber oil.

Rf (Hexane:EtOAc, 4:1): 0.28; ¹H NMR (400 MHz, CDCl₃) δ 8.28 (d, J=9.0 Hz, 2H), 7.37 (d, J=9.0 Hz, 2H), 6.62 (td, J=1.7, 5.2 Hz, 1H), 6.53 (td, J=1.7, 4.8 Hz, 1H), 6.25 (td, J=1.8, 5.5 Hz, 1H), 6.11 (td, J=1.6, 5.1 Hz, 1H), 4.53 (dd, J=7.6, 11.5 Hz, 1H), 4.40 (dd, J=7.4, 11.3 Hz, 1H), 2.52 (quin, J=7.6 Hz, 1H), 1.92 (dd, J=4.7, 8.6 Hz, 1H), 1.76 (dd, J=4.7, 6.7 Hz, 1H) ppm; ¹³C NMR (100 MHz, CDCl₃) δ 155.4, 152.3, 145.3, 138.6, 133.8, 131.7, 129.4, 125.2, 121.7, 70.9, 41.5, 24.6, 16.9 ppm.

### (c) Fmoc-Lys(spiro[2.4]hepta-4,6-dien-1-ylmethyl carbonate)-OH (15)

14 (5.12 g, 17.8 mmol, 1 eq) was added to DMF (40 mL), followed by Fmoc-Lys-OH (7.87 g, 21.4 mmol, 1.2 eq) and DIPEA (7.44 mL, 42.7 mmol, 2.4 eq). The reaction was stirred until consumption of the starting material (NMR, 3.5 hr), then poured into EtOAc (100 mL) and H₂O (140 mL). The aqueous layer was acidified to pH 2-3 with HCl (1 M, 100 mL), poured into a separatory funnel, and the layers separated. The aqueous layer was extracted with EtOAc (2×100 mL). The organic layers were combined, washed with brine (100 mL), dried over Na₂SO₄, filtered, and the solvent removed. The residue was subjected to flash column chromatography (Hexane:EtOAc, 3:1 then DCM:MeOH:AcOH, 89:10:1) and the solvent removed. Residual AcOH and DMF was removed by suspending the product in DCM, washing with brine, drying the organic layer over Na₂SO₄, filtering, then removing the solvent to yield 15 (7.43 g, 81%) as an eggshell foam.

Rf (DCM:MeOH, 90:10): 0.39; ¹H NMR (500 MHz, CDCl₃) δ 8.62 (br. s., 1H), 7.75 (d, J=7.3 Hz, 2H), 7.66-7.49 (m, 2H), 7.39 (t, J=7.4 Hz, 2H), 7.30 (t, J=7.3 Hz, 2H), 6.54 (br. s., 1H), 6.47 (br. s., 1H), 6.21 (br. s., 1H), 6.04 (br. s., 1H), 5.74 (d, J=7.3 Hz, 1H), 4.91 (br. s., 1H), 4.53-4.00 (m, 5H), 3.21-3.00 (m, 2H), 2.97 (s, 1H), 2.90 (d, J=0.8 Hz, 1H), 2.47-2.31 (m, 1H), 1.95-1.27 (m, 6H) ppm; ¹³C NMR (125 MHz, CDCl₃) 163.2, 156.7, 143.6, 141.2, 138.9, 134.5, 130.9, 128.9, 127.6, 127.0, 125.1, 119.9, 115.6, 67.0, 66.5, 53.5, 47.1, 41.6, 40.4, 36.8, 31.8, 29.2, 25.7, 22.2, 21.4, 17.1 δ ppm.

### (d) CP2-NNAA (16)

9 (5.50 g, 10.6 mmol, 1 eq) was added to DMF (150 mL), followed by piperidine (16.8 mL). The reaction was stirred until consumption of the starting material (TLC, 90 min), then the solvent was removed. Et₂O (100 mL) was added to the residue, and the suspension was sonicated for 5 min. The suspension was filtered and rinsed with H₂O (2×100 mL) and Et₂O (100 mL). The solid was suspended in MeOH (10 mL), stirred for 10 min with gentle warming (−40° C.), Et₂O (40 mL) was added, the suspension filtered and rinsed with Et₂O (2×50 mL). The compound was dried under vacuum to yield 10 (2.24 g, 71%) as a white powder.

Rf (DCM:MeOH, 85:15): 0.29; ¹H NMR (400 MHz, DMSO-d₆+1 drop TFA) δ 8.20 (br. s., 3H), 7.16 (t, J=5.5 Hz, 1H), 6.48 (td, J=1.8, 5.1 Hz, 1H), 6.40 (d, J=5.1 Hz, 1H), 6.32 (d, J=5.1 Hz, 1H), 6.12 (td, J=1.9, 4.9 Hz, 1H), 4.24 (dd, J=6.7, 11.7 Hz, 1H), 3.99 (dd, J=7.6, 11.5 Hz, 1H), 3.88 (d, J=5.1 Hz, 1H), 2.94 (d, J=5.9 Hz, 2H), 2.37 (quin, J=7.5 Hz, 1H), 1.83-1.63 (m, 4H), 1.44-1.19 (m, 4H) ppm; ¹³C NMR (100 MHz, DMSO-d₆+1 drop TFA):

171.2, 156.2, 139.3, 135.2, 130.4, 128.3, 65.3, 51.9, 42.0, 29.7, 28.9, 25.7, 21.6, 16.4; MS (EI) Exact mass cald. for C₁₅H₂₂N₂O₄ [M]⁺: 294.1580, found: 294.1571.

### (ii) Synthesis of CP2-NHS (18)

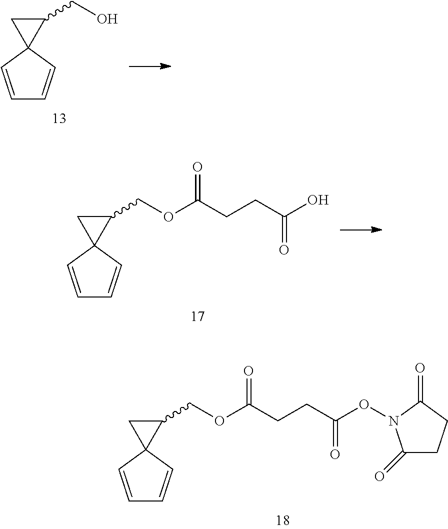 [SMILES: OCC1CC12C=CC=C2 ; O=C(O)CCC(=O)OCC1CC12C=CC=C2 ; O=C(CCC(=O)ON1C(=O)CCC1=O)OCC1CC12C=CC=C2]

### (a) 4-Oxo-4-(spiro[2.4]hepta-4,6-dien-1-ylmethoxy)butanoic acid (17)

DCM (1.5 mL) was added to a vial containing 13 (0.37 g, 3.0 mmol, 1 eq). Et₃N (0.42 mL, 3.0 mmol, 1 eq), DMAP (37 mg, 0.30 mmol, 0.1 eq) and succinic anhydride (0.33 g, 3.3 mmol, 1.1 eq) were added, the reaction capped under an atmosphere of air, and stirred at rt until consumption of the starting material (TLC, 1.75 hr). The reaction mixture was poured into a separatory funnel with DCM (50 mL) and washed with aqueous HCl (1 M, 50 mL). The aqueous layer was extracted with DCM (50 mL), the organic layers combined, dried over Na₂SO₄, filtered, and the solvent removed to yield 17 of sufficient purity for the next reaction.

Rf (EtOAc): 0.56; ¹H NMR (400 MHz, CDCl₃) δ 10.60 (br. s., 1H), 6.57 (td, J=1.9, 5.3 Hz, 1H), 6.50 (td, J=1.8, 5.1 Hz, 1H), 6.21 (td, J=1.7, 5.2 Hz, 1H), 6.07 (td, J=1.8, 5.1 Hz, 1H), 4.37 (dd, J=7.4, 11.7 Hz, 1H), 4.20 (dd, J=7.0, 11.7 Hz, 1H), 2.74-2.57 (m, 4H), 2.42 (quin, J=7.8 Hz, 1H), 1.85 (dd, J=4.5, 8.4 Hz, 1H), 1.69 (dd, J=4.3, 7.0 Hz, 1H) ppm.

### (b) CP2-NHS (18)

THF (10 mL) was added to a vial containing 17 (theo 3.0 mmol, 1 eq). NHS (0.48 g, 4.2 mmol, 1.4 eq), EDC-HCl (0.69 g, 3.6 mmol, 1.2 eq) and DCM (5 mL) were added, the reaction capped under an atmosphere of air, and stirred at rt overnight. The solvent was removed and the residue was subjected to flash column chromatography (Hexane:EtOAc, 1:1) to yield 18 (0.59 g, 62% over two steps) as a colourless, viscous oil.

Rf (Hexane:EtOAc, 1:1): 0.34; ¹H NMR (400 MHz, CDCl₃) δ 6.56 (td, J=1.8, 5.1 Hz, 1H), 6.48 (td, J=1.8, 5.1 Hz, 1H), 6.21 (td, J=1.6, 3.4 Hz, 1H), 6.06 (td, J=1.6, 3.4 Hz, 1H), 4.36 (dd, J=7.4, 11.7 Hz, 1H), 4.21 (dd, J=7.4, 11.7 Hz, 1H), 2.93 (t, J=7.0 Hz, 2H), 2.83 (s, 4H), 2.73 (t, J=7.4 Hz, 2H), 2.42 (quin, J=7.6 Hz, 1H), 1.83 (dd, J=4.3, 8.6 Hz, 1H), 1.68 (dd, J=4.5, 6.8 Hz, 1H) ppm; ¹³C NMR (100 MHz, CDCl₃) δ 170.8, 168.9, 167.6, 138.8, 134.3, 131.2, 129.0, 66.6, 41.5, 28.6, 26.2, 25.5, 25.1, 17.3 ppm.

### Example 5. CP2 Diene-Maleimide Conjugation for Preparation of ADCs Via Crosslinker-Modified mAb

The feasibility of spirocyclopentadiene-maleimide reactions for bioconjugation was evaluated. Spirocyclopentadiene groups were introduced via an amine-reactive heterobifunctional linker.

Introduction of CP2 functionality onto mAbs: CP2 diene functionality was installed onto IgG1 mAbs by reaction of lysine primary amines with NHS-ester activated CP2 diene. This approach resulted in randomly conjugated, amide-linked cyclopentadiene groups. The resulting antibody is termed mAb-CP2-linker, but may also be denoted as mAb-CP2 in figures. See figure captions for clarification. A typical mAb modification reaction is described as follows. Mab solution was adjusted to 5 mg/mL (3 mL, 15 mg mAb, 100 nmol, 1 eq.) with PBS pH 7.2 followed by addition of 10% v/v 1 M NaHCO₃. This solution was chilled on ice and 35 μL CP2-NHS (10 mM stock in DMAc, 350 nmol, 3.5 equivalents) was added. The reaction proceeded on ice for 5 minutes followed by reaction at room temperature for 1 h with continuous mixing. Reacted mAb was purified by dialysis (Slide-A-Lyzer, 10 kDa MWCO) against PBS, 1 mM EDTA, pH 7.4, 0° C. for 24 h. CP2 introduction was quantified by intact deglycosylated mass spectrometry as described below and found to be 3.29 CP2-linkers (and thus dienes) per mAb in this example, which corresponds to 94% conversion of CP2-NHS to antibody conjugate.

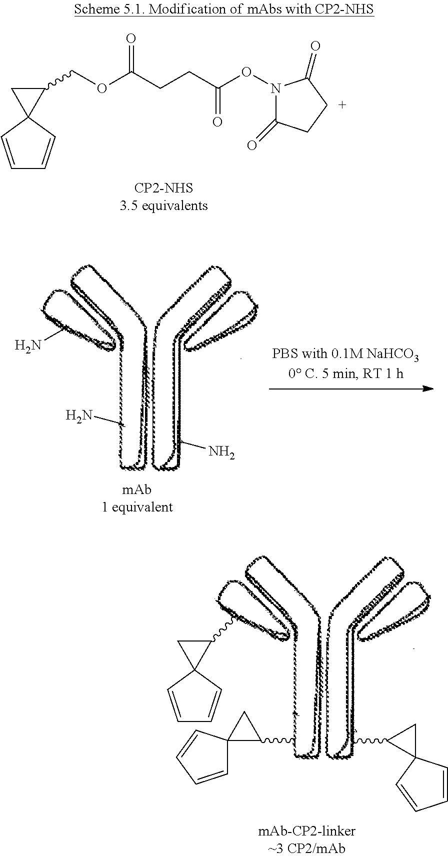

Mass spectrometry analysis: First, mAbs or mAb conjugates were deglycosylated with EndoS (New England BioLabs) by combining 50 μL sample (1 mg/mL mAb) with 5 μL glyco buffer 1 (New England BioLabs) and 5 μL Remove-iT EndoS (1:10 dilution in PBS, 20,000 units/mL, New England BioLabs) followed by incubation for 1 h at 37° C. Reduced samples were prepared by addition of 5 μL Bond-Breaker TCEP solution (0.5 M, Thermo Fisher Scientific) and incubation for 10 min at 37° C. Mass spectrometry analysis was performed using an Agilent 6520B Q-TOF mass spectrometer equipped with a RP-HPLC column (ZORBAX 300 Diphenyl RRHD, 1.8 micron, 2.1 mm×50 mm). High-performance liquid chromatography (HPLC) parameters were as follows: flow rate, 0.5 ml/min; mobile phase A was 0.1% (v/v) formic acid in HPLC-grade H₂O, and mobile phase B was 0.1% (v/v) formic acid in acetonitrile. The column was equilibrated in 90% A/10% B, which was also used to desalt the mAb samples, followed by elution in 20% A/80% B. Mass spec data were collected for 100-3000 m/z, positive polarity, a gas temperature of 350° C., a nebulizer pressure of 48 lb/in², and a capillary voltage of 5,000 V. Data were analyzed using vendor-supplied (Agilent v.B.04.00) MassHunter Qualitative Analysis software and peak intensities from deconvoluted spectra were used to derive the relative proportion of species in each sample.

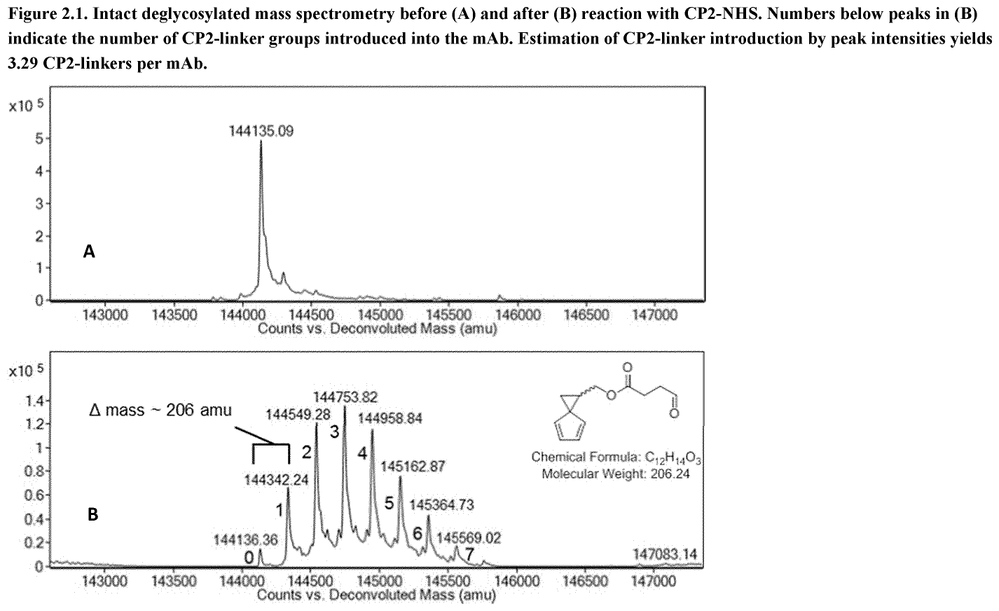

FIG. 2.1. Intact deglycosylated mass spectra before (A) and after (B) reaction with CP2-NHS. Numbers below peaks in (B) indicate the number of CP2-diene groups introduced into the mAb. Estimation of CP2-linker introduction by peak intensities yields 3.29 CP2-dienes per mAb.

TABLE 5.1
Summary of CP2-NHS mAb reaction

| equivalents CP1-NHS | [mAb] | CP2-linker | conversion |
|---|---|---|---|
| (rel to mAb) | mg/mL | per mAb | (%) |
| 3.5 | 5 | 3.29 | 94 |

### Example 6. CP2-NNAA Incorporation into Antibodies

Incorporation of CP2-NNAA into position K274 or S239 of an anti EphA2 (1C1) antibody, the quality of expressed mAb, and reactivity of CP2-NNAA diene after antibody incorporation was assessed.

Preparation of CP2 NNAA stock solution: CP2 NNAA (0.5 g, 1.7 mmol) was combined with 7.8 mL 0.2 M NaOH in H₂O. The resulting slurry was stirred at room temperature until all solids dissolved (10 minutes). After complete dissolution the light-yellow solution was passed through a 0.2 m filter, aliquoted, and stored at −80° C. until use. This procedure resulted in 8.2 mL of 216 mM CP2 NNAA stock solution.

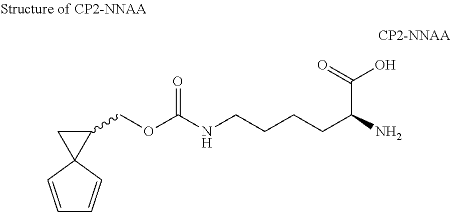 [SMILES: N[C@@H](CCCCNC(=O)OCC1CC12C=CC=C2)C(=O)O]

Antibody expression: 12G3H11 or 1C1 IgG1 antibody genes with an amber mutation at Fc position K274 or S239 were cloned into a proprietary pOE antibody expression vector. The construct was transfected into CHO-G22 by PEImax (1.5 L of G22 cells), along with a plasmid encoding PyIRS double mutant (Y306A/Y384F) or wild-type PyIRS and a plasmid containing tandem repeats of the tRNA expression cassette (pORIP 9× tRNA). Four hours post transfection, 3.3% of feed F9 (proprietary) and 0.2% of feed F10 (proprietary) were added to cells and the cells were further incubated at 34 degrees. CP2-NNAA was added the next day at final concentration of 0.26 mM for 1C1 K274 and 1C1 S239 transfected cells. Cells were fed again on day 3 and day 7 with 6.6% of feed F9 and 0.4% of feed F10. Cells were spun down and supernatant was harvested on day 11. The supernatant was purified by IgSelect affinity column (GE Health Care Life Science). The antibody was eluted with 50 mM glycine, 30 mM NaCl, pH 3.5 elution buffer, neutralized with 1 M Tris buffer pH 7.5, and dialyzed into PBS, pH 7.2. Concentration of antibody eluted was determined by absorbance measurement at 280 nm. The back calculated titer was 57 mg/L for 1C1 K274CP2-NNAA and 76 mg/L for 1C1 S239CP2-NNAA. 12G3H11 mAb was expressed in a similar manner at smaller scale, with CP2-NNAA feed concentration varied. Recovered antibody was analyzed by SDS-PAGE using standard methods. Antibody was also analyzed by size exclusion chromatography and mass spectrometry as described below. Antibodies incorporating CP2-NNAA are denoted as mAb-CP1-NNAA to distinguish them from mAb-CP2-linker constructs, or mAb-[position]CP2-NNAA where [position] indicates the amino acid number and amino acid symbol that was mutated to CP2-NNAA.

Size exclusion chromatography: SEC analysis was performed using an Agilent 1100 Capillary LC system equipped with a triple detector array (Viscotek 301, Viscotek, Houson, Tex.); the wavelength was set to 280 nm, and samples were run on a TSK-GEL G3000SWXL column (Toso Bioscience LLC, Montgomeryville, Pa.) using 100 mM sodium phosphate buffer, pH 6.8 at a flow rate of 1 mL/min.

Mass spectrometry analysis: For deglycosylated mAb analysis, EndoS (5 μL Remove-iT EndoS (1:10 dilution in PBS, 20,000 units/mL, New England BioLabs) was combined with 50 μL sample (1 mg/mL mAb) and 5 μL glyco buffer 1 (New England BioLabs) and followed by incubation for 1 h at 37° C. Reduced samples were prepared by addition of 5 μL Bond-Breaker TCEP solution (0.5 M, Thermo Fisher Scientific) and incubation for 10 min at 37° C. Mass spectrometry analysis was performed using an Agilent 6520B Q-TOF mass spectrometer equipped with a RP-HPLC column (ZORBAX 300 Diphenyl RRHD, 1.8 micron, 2.1 mm×50 mm). High-performance liquid chromatography (HPLC) parameters were as follows: flow rate, 0.5 ml/min; mobile phase A was 0.1% (v/v) formic acid in HPLC-grade H₂O, and mobile phase B was 0.1% (v/v) formic acid in acetonitrile. The column was equilibrated in 90% A/10% B, which was also used to desalt the mAb samples, followed by elution in 20% A/80% B. Mass spec data were collected for 100-3000 m/z, positive polarity, a gas temperature of 350° C., a nebulizer pressure of 48 lb/in², and a capillary voltage of 5,000 V. Data were analyzed using vendor-supplied (Agilent v.B.04.00) MassHunter Qualitative Analysis software and peak intensities from deconvoluted spectra were used to derive the relative proportion of species in each sample.

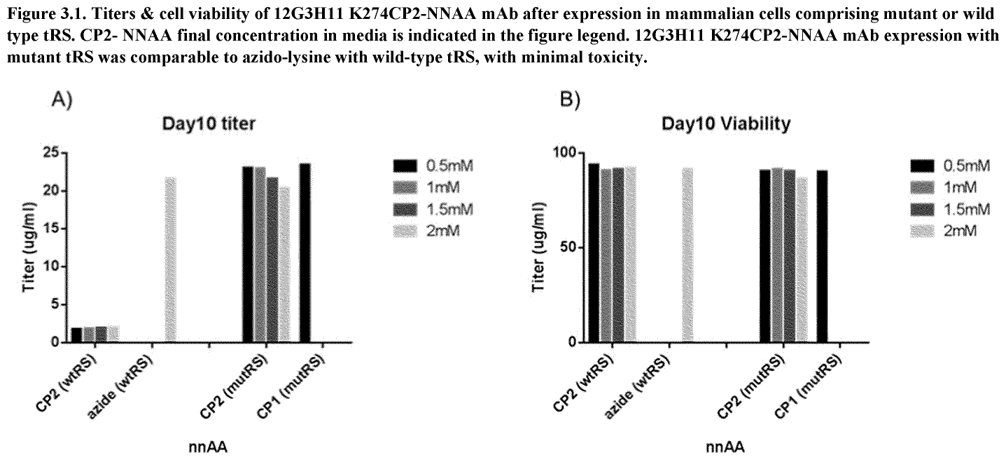

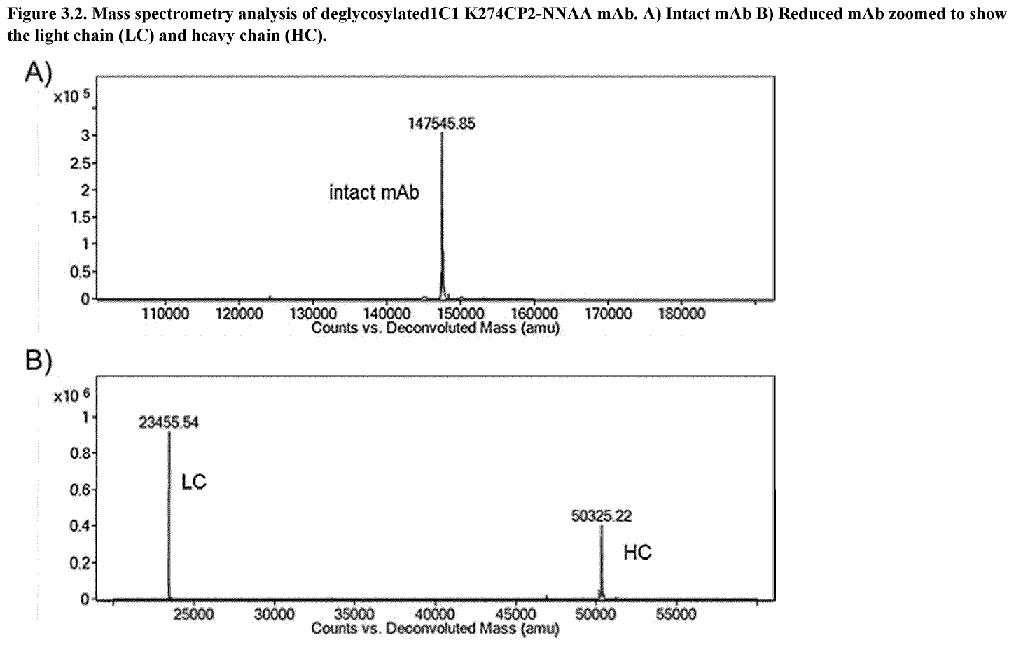

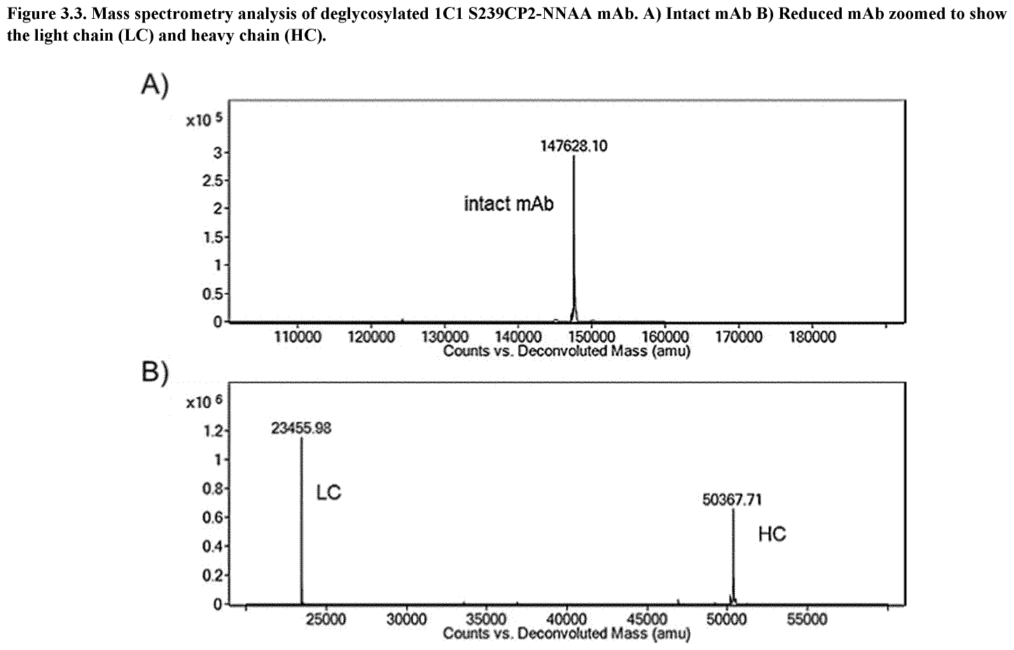

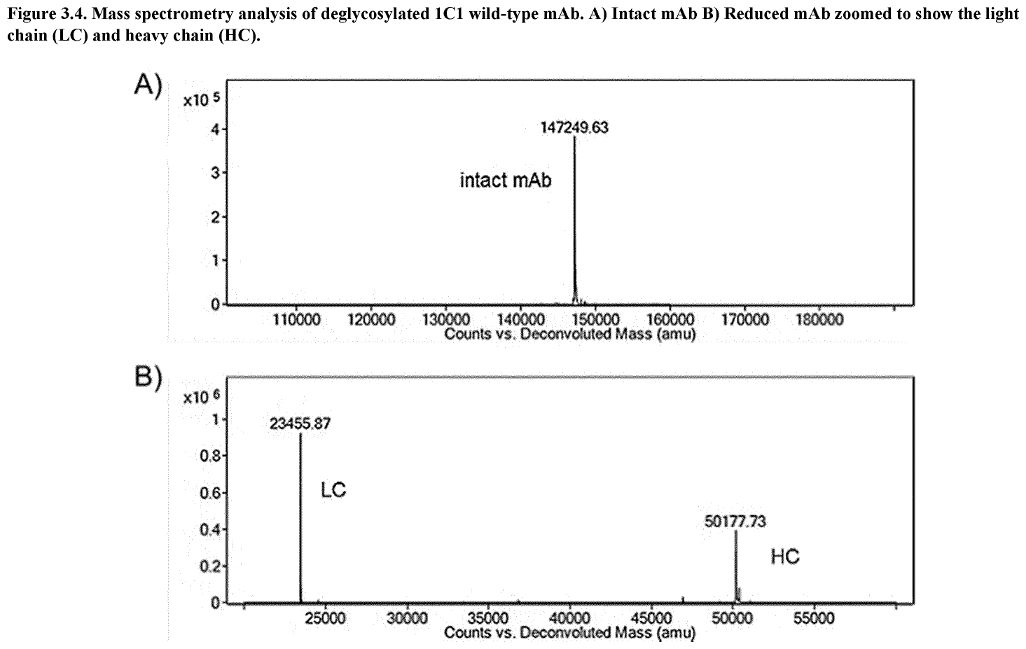

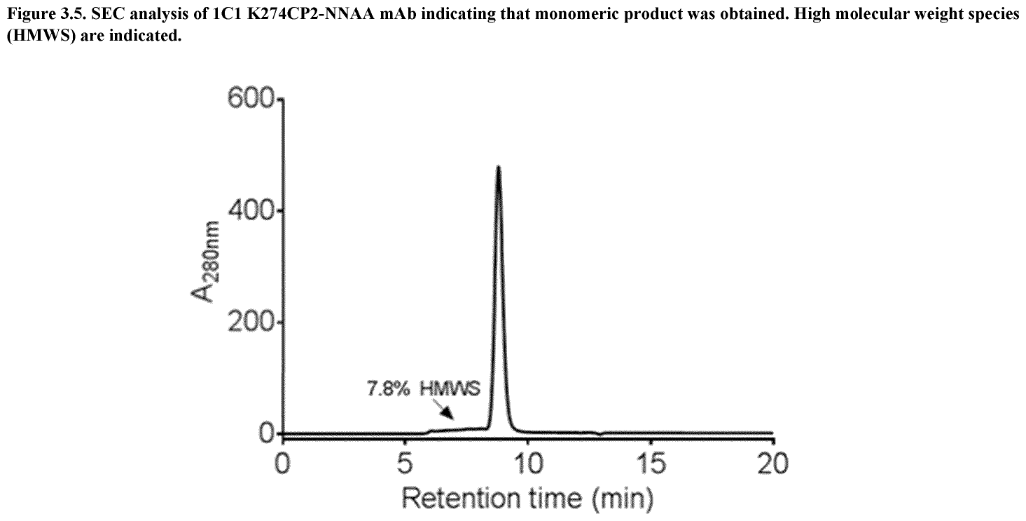

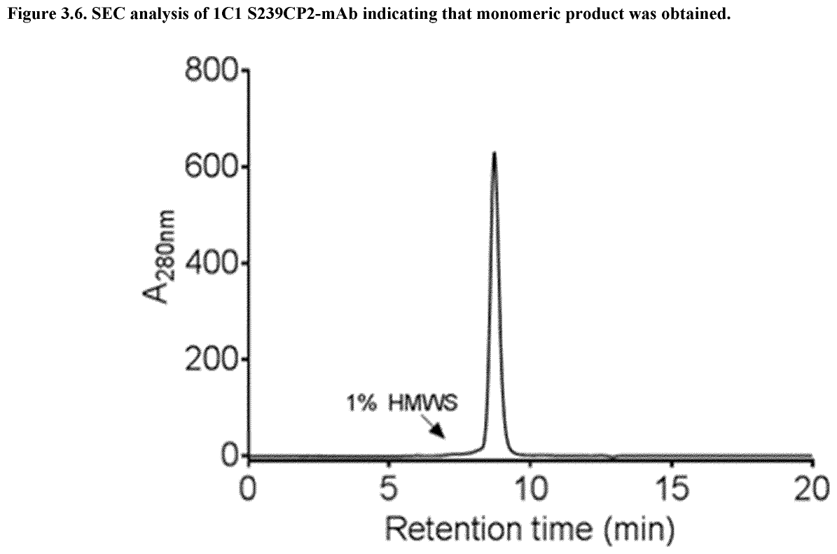

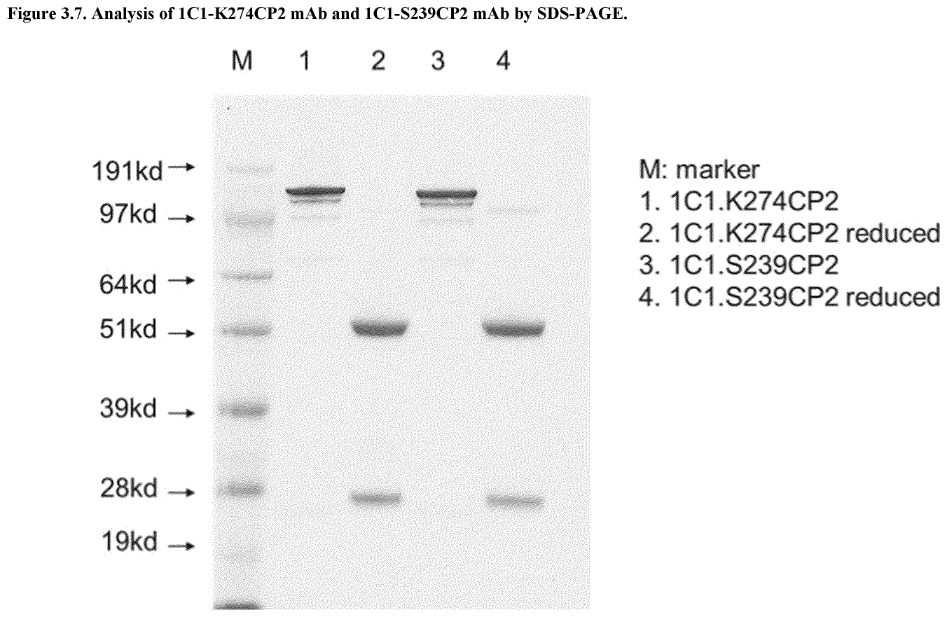

FIG. 3.1. Titers and cell viability of 12G3H11 K274CP2-NNAA mAb after expression in mammalian cells comprising mutant or wild type tRS. CP2-NNAA final concentration in media is indicated in the figure legend. 12G3H11 K274CP2-NNAA mAb expression with mutant tRS was comparable to azido-lysine with wild-type tRS, with minimal toxicity.

TABLE 6.1
Summary of 1C1 K274CP2-NNAA and
1C1 S239CP2-NNAA mAb production

| | K274 | S239 |
|---|---|---|
| NNAA feed (mM) | 0.5 | 0.5 |
| Volume (L) | 2 | 2 |
| Mass recovered (mg) | 114 | 153 |
| Titer (mg/L) | 57 | 76 |
| Monomer (%) | 93.2 | 99 |

FIG. 3.2. Mass spectrometry analysis of deglycosylated1C1 K274CP2-NNAA mAb. A) Intact mAb B) Reduced mAb zoomed to show the light chain (LC) and heavy chain (HC). The observed intact mass closely matched the calculated intact mass (147546.03) assuming incorporation of two CP2-NNAAs in the intact mAb structure. The observed heavy chain mass closely matched the calculated heavy chain mass (50325.93) assuming incorporation of one CP2-NNAA into the antibody heavy chain. No incorporation of CP2-NNAA into the mAb light chain was observed. Analogous spectra for 1C1 wild-type mAb are shown in FIG. 3.4.

FIG. 3.3. Mass spectrometry analysis of deglycosylated 1C1 S239CP2-NNAA mAb. A) Intact mAb B) Reduced mAb zoomed to show the light chain (LC) and heavy chain (HC). The observed intact mass closely matched the calculated intact mass (147628.23) assuming incorporation of two CP2 amino acids in the intact mAb structure. The observed heavy chain mass closely matched the calculated heavy chain mass (50367.03) assuming incorporation of CP2-NNAA into the antibody heavy chain. No incorporation of CP2-NNAA into the mAb light chain was observed. Analogous spectra for 1C1 wild-type mAb are shown in FIG. 3.4

FIG. 3.4. Mass spectrometry analysis of deglycosylated 1C1 wild-type mAb. A) Intact mAb B) Reduced mAb zoomed to show the light chain (LC) and heavy chain (HC). A) Mass range showing intact mAb, B) mass range showing light chain (LC) and heavy chain (HC).

TABLE 6.2
Summary of mass spectrometry data for 1C1-
K274CP2-NNAA and 1C1-S239CP2-NNAA mAbs

| | K274 | S239 | WT |
|---|---|---|---|
| Observed intact mass | 147545.85 | 147628.1 | 147249.63 |
| Observed change relative to WT | +296.2 | +378.4 | NA |
| Calculated change relative to WT | +296.4 | +378.6 | NA |
| Observed heavy chain mass | 50325.22 | 50367.71 | 50177.73 |
| Observed change relative to WT | +147.5 | +189.9 | NA |
| Calculated change relative to WT | +148.2 | +189.3 | NA |

FIG. 3.5. SEC analysis of 1C1 K274CP2-NNAA mAb indicating that monomeric product was obtained. High molecular weight species (HMWS) are indicated.

FIG. 3.6. SEC analysis of 1C1 S239CP2-NNAA mAb indicating that monomeric product was obtained.

FIG. 3.7. Analysis of 1C1-K274CP2-NNAA mAb and 1C1-S239CP2-NNAA mAb by SDS-PAGE.

Incorporation of CP2-NNAA into antibodies at positions K274 and S239 was confirmed by mass spectrometry. Recovered antibody was of high quality, with no truncated product and very little aggregate. Titers achieved at 2 L production scale for 1C1 antibody were reasonably high considering the low amount of CP2-NNAA fed to cells.

### Example 7—Antibody-Drug Conjugate with Compound 10: ConjA

Compound 10 was added as a DMSO solution (3 molar equivalent/antibody, 0.2 micromole, in 0.5 mL DMSO) to 4.0 mL of the 1C1S239CP2 antibody solution in PBS, 1 mM EDTA, pH 7.4 (10.0 mg, 66.7 nanomoles) and 0.5 mL of 1M sodium phosphate pH 6.0 for a 10% (v/v) final DMSO concentration and a final pH of 6.0. The solution was left to react at room temperature for overnight with gentle shaking. The conjugation was quenched by the addition of N-acetyl cysteine (3.3 micromoles, 33 μL at 100 mM), and purified by preparative size exclusion chromatography using FPLC and Superdex 200 26/600 column with PBS pH 7.4 as elution buffer. Fractions containing over 95% monomers were pooled, concentrated, buffer exchanged to 25 mM Histidine, 200 mM Sucrose, pH 6.0 by spin filtration using 15 mL Amicon Ultracell 50 kDa MWCO spin filter, sterile filtered and analysed.

UHPLC analysis on a Shimadzu Prominence system using a Proteomix HIC Butyl-NP5, 5 μm, non-porous, 4.6×35 mm (Sepax) column eluting with a gradient of 1.5M ammonium sulphate, 25 mM sodium acetate, pH 7.4 and 25 mM sodium acetate, pH 7.4 with 20% acetonitrile (v/v) on a neat sample of ConjA at 214 nm shows unconjugated antibody and a mixture of singly conjugated and doubly conjugated Compound 10, consistent with a drug-per-antibody ratio (DAR) of 0.95 molecules of Compound 10 per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a neat sample of ConjA at 280 nm shows a monomer purity of 99%. UHPLC SEC analysis gives a concentration of final ConjA at 1.95 mg/mL in 3.9 mL, obtained mass of ConjA is 7.6 mg (76% yield).

### Example 8—Antibody-Drug Conjugate with Compound 11: ConjB

Compound 11 was added as a DMSO solution (1.5 molar equivalent/antibody, 0.1 micromole, in 0.5 mL DMSO) to 4.0 mL of the 1C1S239CP2 antibody solution in PBS, 1 mM EDTA, pH 7.4 (10.0 mg, 66.7 nanomoles) and 0.5 mL of 1M sodium phosphate pH 6.0 for a 10% (v/v) final DMSO concentration and a final pH of 6.0. The solution was left to react at room temperature for overnight with gentle shaking. The conjugation was quenched by the addition of N-acetyl cysteine (1.7 micromoles, 17 μL at 100 mM), and purified by preparative size exclusion chromatography using FPLC and Superdex 200 26/600 column with PBS pH 7.4 as an elution buffer. Fractions containing over 95% monomers were pooled, concentrated, buffer exchanged to 25 mM Histidine, 200 mM Sucrose, pH 6.0 by spin filtration using 15 mL Amicon Ultracell 50 kDa MWCO spin filter, sterile filtered and analysed.

UHPLC analysis on a Shimadzu Prominence system using a Proteomix HIC Butyl-NP5, 5 μm, non-porous, 4.6×35 mm (Sepax) column eluting with a gradient of 1.5M ammonium sulphate, 25 mM sodium acetate, pH 7.4 and 25 mM sodium acetate, pH 7.4 with 20% acetonitrile (v/v) on a neat sample of ConjB at 214 nm shows unconjugated antibody and a mixture of singly conjugated and doubly conjugated Compound 11, consistent with a drug-per-antibody ratio (DAR) of 0.97 molecules of Compound 11 per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of ConjB at 280 nm shows a monomer purity of 99%. UHPLC SEC analysis gives a concentration of final ConjB at 1.97 mg/mL in 4.0 mL, obtained mass of ConjB is 7.9 mg (79% yield).

### Example 9—Antibody-Drug Conjugate with Compound 12: ConjC

Compound 12 was added as a DMSO solution (5 molar equivalent/antibody, 0.17 micromole, in 0.5 mL DMSO) to 1.75 mL of the 1C1S239CP2 antibody solution in PBS, 1 mM EDTA, pH 7.4 (5.0 mg, 33.3 nanomoles) and 0.25 mL of 1M sodium phosphate pH 6.0 for a 10% (v/v) final DMSO concentration and a final pH of 6.0. The solution was left to react at 37° C. for 48 hrs with gentle shaking. The conjugation was quenched by the addition of N-acetyl cysteine (1.67 micromoles, 16.7 μL at 100 mM), and purified by hydrophobic interaction chromatography using FPLC and HP-Butyl column (5 mL) with a gradient run of 1 M (NH4)₂SO₄, 25 mM Potassium Phosphate pH 6.0, and 25 mM Potassium Phosphate pH 6.0. Fractions containing over 95% DAR1 were pooled, concentrated, buffer exchanged to 25 mM Histidine, 200 mM Sucrose, pH 6.0 by spin filtration using 15 mL Amicon Ultracell 50 kDa MWCO spin filter, sterile filtered and analysed.

UHPLC analysis on a Shimadzu Prominence system using a Proteomix HIC Butyl-NP5, 5 μm, non-porous, 4.6×35 mm (Sepax) column eluting with a gradient of 1.5M ammonium sulphate, 25 mM sodium acetate, pH 7.4 and 25 mM sodium acetate, pH 7.4 with 20% acetonitrile (v/v) on a neat sample of ConjC at 214 nm shows a small fraction of unconjugated antibody and mostly singly conjugated Compound 12, consistent with a drug-per-antibody ratio (DAR) of 0.96 molecules of Compound 12 per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of ConjC at 280 nm shows a monomer purity of 98%. UHPLC SEC analysis gives a concentration of final ConjC at 1.20 mg/mL in 0.75 mL, obtained mass of ConjC is 0.9 mg (18% yield).

### Example 10—Antibody-Drug Conjugate with Compound X: ConjD

### Compound X:

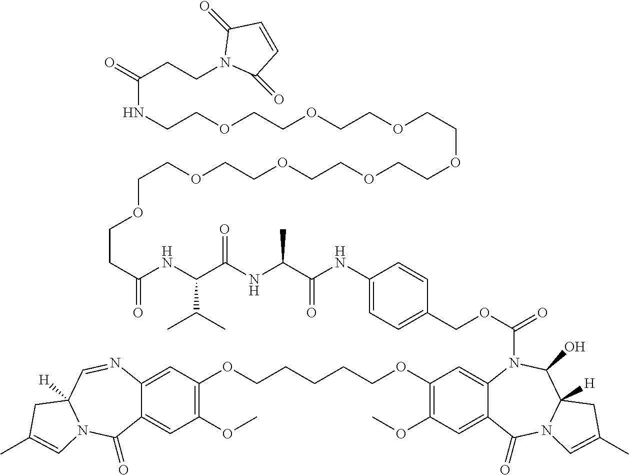

is compound B of WO2014/057074.

Compound X was added as a DMSO solution (10 molar equivalent/antibody, 0.67 micromole, in 0.43 mL DMSO) to 4.5 mL of the 1 C1 S239CP2 antibody solution in PBS, 1 mM EDTA, pH 7.4 (10.0 mg, 66.7 nanomoles) for a 10% (v/v) final DMSO concentration. The solution was left to react at room temperature for overnight with gentle shaking. The conjugation was quenched by the addition of N-acetyl cysteine (3.3 micromoles, 33 μL at 100 mM), and purified by preparative size exclusion chromatography using FPLC and Superdex 20026/600 column with PBS pH 7.4 as elution buffer. Fractions containing over 95% monomers were pooled, concentrated, buffer exchanged to 25 mM Histidine, 200 mM Sucrose, pH 6.0 by spin filtration using 15 mL Amicon Ultracell 50 kDa MWCO spin filter, sterile filtered and analysed.

UHPLC analysis on a Shimadzu Prominence system using a Proteomix HIC Butyl-NP5, 5 μm, non-porous, 4.6×35 mm (Sepax) column eluting with a gradient of 1.5M ammonium sulphate, 25 mM sodium acetate, pH 7.4 and 25 mM sodium acetate, pH 7.4 with 20% acetonitrile (v/v) on a neat sample of ConjD at 214 nm shows unconjugated antibody and a mixture of singly conjugated and doubly conjugated Compound X, consistent with a drug-per-antibody ratio (DAR) of 1.91 molecules of Compound X per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a neat sample of ConjD at 280 nm shows a monomer purity of 99%. UHPLC SEC analysis gives a concentration of final ConjD at 1.49 mg/mL in 4.0 mL, obtained mass of ConjD is 6.0 mg (60% yield).

### Example 11—Cytotoxicity Assays for ADCs

Medium from sub-confluent (80-90% confluency) PC3 cells in a T75 flask was aspirated and the flask rinsed with PBS (about 20 ml) and emptied. Trypsin-EDTA (5 ml) was added, the flask returned to the 37° C. gassed incubator for up to about 5 minutes, then rapped sharply to dislodge and dissociate cells from the plastic. The cell suspension was transferred to a sterile 50 ml screw-top centrifuge tube, diluted with growth medium to a final volume of 15 ml, then centrifuged (400 g for 5 min). The supernatant was aspirated and the pellet re-suspended in 10 ml culture medium. Repeated pipetting may be necessary to produce monodisperse cell suspensions. The cell concentration and viability are measured of trypan blue cell stained cells, using the LUNA II. Cells were diluted to 1500 cells/well, dispensed (50 μl/well) into white 96 well flat bottom plates and incubated overnight before use.

A stock solution (1 ml) of antibody drug conjugate (ADC) (20 μg/ml) was made by dilution of filter-sterilised ADC into cell culture medium. A set of 8× 10-fold dilutions of stock ADC were made in a 24 well plate by serial transfer of 100 μl onto 900 μl of cell culture medium. ADC dilution was dispensed (50 μl/well) into 4 replicate wells of the 96-well plate, containing 50 μl cell suspension seeded the previous day. Control wells received 50 μl cell culture medium. The 96-well plate containing cells and ADCs was incubated at 37° C. in a CO₂-gassed incubator for 6 days. At the end of the incubation period, plates were equilibrated to room temperature for 30 min before CellTiter-Glo (Promega) was dispensed (100 μl per well) into each well. Plates were placed on an orbital shaker for 2 min before stabilisation at room temperature for 10 min. Well luminescence was measured and percentage cell survival was calculated from the mean luminescence in the 4 ADC-treated wells compared to the mean luminescence in the 4 control untreated wells (100%). IC₅₀ was determined from the dose-response data using GraphPad Prism using the non-linear curve fit algorithm: sigmoidal dose response, X is log(concentration). Cell growth medium for PC3 was: F12K with glutamine, 10% (v/v) HyClone™ Fetal Bovine Serum.

| | EC₅₀ (μg/ml) |
|---|---|
| ConjA | 0.001566 |
| ConjB | 0.001839 |
| ConjC | 0.01095 |
| ConjD | 0.006663 |

All documents and other references mentioned above are herein incorporated by reference.

1.-113. (canceled)

114. A conjugate of formula I: 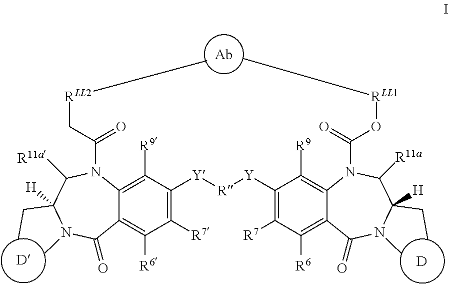
wherein
Ab is a modified antibody having at least one free conjugation site on each heavy chain
D represents either group D1 or D2: 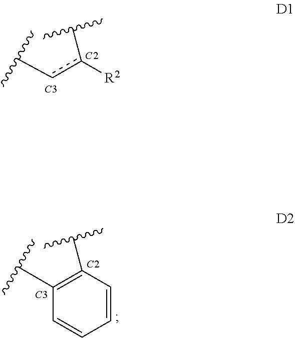
the dotted line indicates the optional presence of a double bond between C2 and C3;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id) 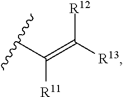
wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(ie) R 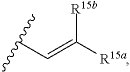
wherein one of R^{15a} and R^{15b} is H and the other is selected from: phenyl, which phenyl is optionally substituted by a group selected from halo, methyl, methoxy; pyridyl; and thiophenyl; and
(if) 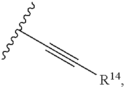
where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C₂ and C3,
R² is selected from H, OH, F, diF and 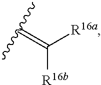
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
D′ represents either group D′1 or D′2: 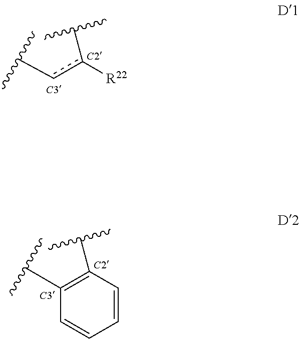
wherein the dotted line indicates the optional presence of a double bond between C2′ and C3′;
when there is a double bond present between C2′ and C3′, R²² is selected from the group consisting of:
(iia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl;
(iid) 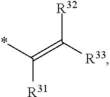
wherein each of R³¹, R³² and R³³ are independently selected from H, C₁₋₃ saturated alkyl, C₂₋₃ alkenyl, C₂₋₃ alkynyl and cyclopropyl, where the total number of carbon atoms in the R²² group is no more than 5;
(iie) 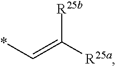
wherein one of R^{25a} and R^{25b} is H and the other is selected from: phenyl, which phenyl is optionally substituted by a group selected from halo, methyl, methoxy; pyridyl; and thiophenyl; and
(iif) 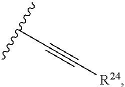
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R²² is selected from H, OH, F, diF and 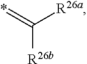
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{11a} is selected from OH, OR^{A}, where R^{A} is C₁₋₄ alkyl;
R^{6′}, R^{7′}, R^{9′} and R^{11a′} are selected from the same groups as R⁶, R⁷, R⁹ and R^{11a} respectively; and
R^{LL1} and R^{LL2} are linkers connected to the antibody at different sites which are independently selected from: 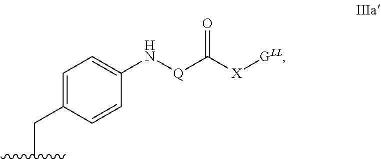
wherein
Qis: 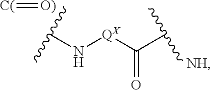
where Q^{X} is such that Q is an amino-acid residue, a dipeptide residue or a tripeptide residue;
X is: 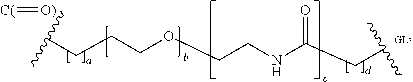
where a=0 to 5, b=0 to 16, c=0 or 1, d=0 to 5;
G^{LL} is a linker connected to the antibody comprising the group:

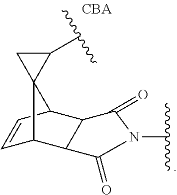 [SMILES: *C1CC12C1C=CC2C2C(=O)N(*)C(=O)C21 ; "."]

115. A conjugate according to claim 114, wherein:
a) both Y and Y′ are O;
b) R″ is a C₃₋₇ alkylene or a group of formula: 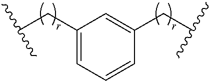
where r is 1 or 2;
c) R⁹ is H.
d) R⁶ is H.
e) R⁷ is selected from H, OH and OR a C₁₋₄ alkyloxy group.

116. A conjugate according to claim 114, wherein D is D1, there is a double bond between C2 and C3, and R² is:
a) a C₅₋₇ aryl group, wherein R² optionally bears one to three substituent groups selected from methoxy, ethoxy, fluoro, chloro, cyano, bis-oxy-methylene, methyl-piperazinyl, morpholino and methyl-thiophenyl;
b) a C₈₋₁₀ aryl group wherein R² optionally bears one to three substituent groups selected from methoxy, ethoxy, fluoro, chloro, cyano, bis-oxy-methylene, methyl-piperazinyl, morpholino and methyl-thiophenyl;
c) a C₁₋₅ saturated aliphatic alkyl group optionally wherein R² is methyl, ethyl or propyl;
d) a C₃₋₆ saturated cycloalkyl group optionally wherein R² is cyclopropyl;
e) a group of formula: 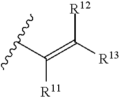
optionally wherein:
i) the total number of carbon atoms in the R² group is no more than 4;
ii) the total number of carbon atoms in the R² group is no more than 3;
iii) one of R¹¹, R¹² and R¹³ is H, with the other two groups being selected from H, C₁₋₃ saturated alkyl, C₂₋₃ alkenyl, C₂₋₃ alkynyl and cyclopropyl;
iv) two of R¹¹, R¹² and R¹³ are H, with the other group being selected from H, C₁₋₃ saturated alkyl, C₂₋₃ alkenyl, C₂₋₃ alkynyl and cyclopropyl;
f) a group of formula: 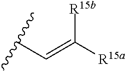
optionally wherein R² is the group: 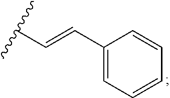 [SMILES: */C=C/c1ccccc1 ; ";"]
g) a group of formula: 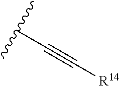
wherein R¹⁴ is selected from H, methyl, ethyl, ethenyl and ethynyl.

117. A conjugate according to claim 114, wherein D is D1, there is a single bond between C2 and C3, and R₂ is:
a) H;
b) 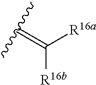
and R^{16a} and R^{16b} are both H;
c) 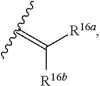
and R^{16a} and R^{16b} are both methyl;
d) 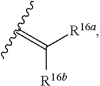
one of R^{16a} and R^{16b} is H, and the other is selected from C₁₋₄ saturated alkyl, C₂₋₃ alkenyl, which alkyl and alkenyl groups are optionally substituted.

118. A conjugate according to claim 114, wherein D′ is D′1, there is a double bond between C2′ and C3′, and R²² is:
a) a C₅₋₇ aryl group wherein R²² optionally bears one to three substituent groups selected from methoxy, ethoxy, fluoro, chloro, cyano, bis-oxy-methylene, methyl-piperazinyl, morpholino and methyl-thiophenyl;
b) a C₈₋₁₀ aryl group wherein R²² optionally bears one to three substituent groups selected from methoxy, ethoxy, fluoro, chloro, cyano, bis-oxy-methylene, methyl-piperazinyl, morpholino and methyl-thiophenyl;
c) a C₁₋₅ saturated aliphatic alkyl group optionally wherein R²² is methyl, ethyl or propyl;
d) a C₃₋₆ saturated cycloalkyl group, optionally wherein R²² is cyclopropyl;
e) a group of formula: 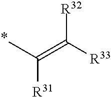
optionally wherein:
i) the total number of carbon atoms in the R²² group is no more than 4;
ii) the total number of carbon atoms in the R²² group is no more than 3;
iii) one of R³¹, R³² and R³³ is H, with the other two groups being selected from H, C₁₋₃ saturated alkyl, C₂₋₃ alkenyl, C₂₋₃ alkynyl and cyclopropyl;
iv) two of R³¹, R³² and R³³ are H, with the other group being selected from H, C₁₋₃ saturated alkyl, C₂₋₃ alkenyl, C₂₋₃ alkynyl and cyclopropyl;
f) a group of formula: 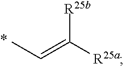
optionally wherein R²² is the group: 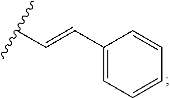 [SMILES: */C=C/c1ccccc1 ; ";"]
g) a group of formula: 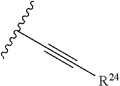
optionally wherein R²⁴ is selected from H, methyl, ethyl, ethenyl and ethynyl.

119. A conjugate according to claim 114, wherein D′ is D′1, there is a single bond between C2′ and C3′, and R²² is:
a) H;
b) 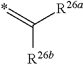
and R^{26a} and R^{26b} are both H;
c) 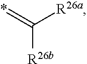
and R^{26a} and R^{26b} are both methyl;
d) 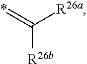
one of R^{26a} and R^{26b} is H, and the other is selected from C₁₋₄ saturated alkyl, C₂₋₃ alkenyl, which alkyl and alkenyl groups are optionally substituted.

120. A conjugate according to claim 114, wherein R^{11a} is
a) OH; or
b) OR^{A}, where R^{A} is C₁₋₄ alkyl.

121. A conjugate according to claim 114, wherein:
a) R^{6′} is selected from the same groups as R⁶, R^{7′} is selected from the same groups as R⁷, R^{9′} is selected from the same groups as R⁹, R^{11a′} is selected from the same groups as R^{11a} and Y′ is selected from the same groups as Y; and/or
b) R^{6′} is the same group as R⁶, R^{7′} is the same group as R⁷, R^{9′} is the same group as R⁹, R^{11a′} is the same group as R^{11a} and Y′ is the same group as Y; and/or
c) R²² is the same group as R².

122. A conjugate according to claim 114, which is of formula Ia-1, Ia-2 or Ia-3: 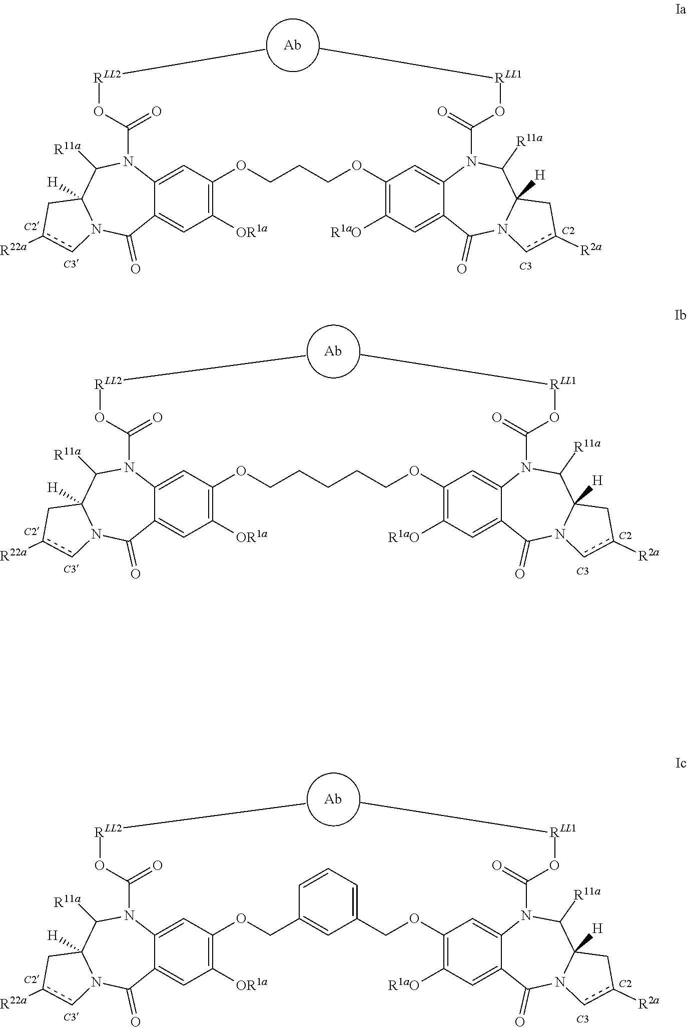
where R^{2a} and R^{22a} are the same and are selected from: 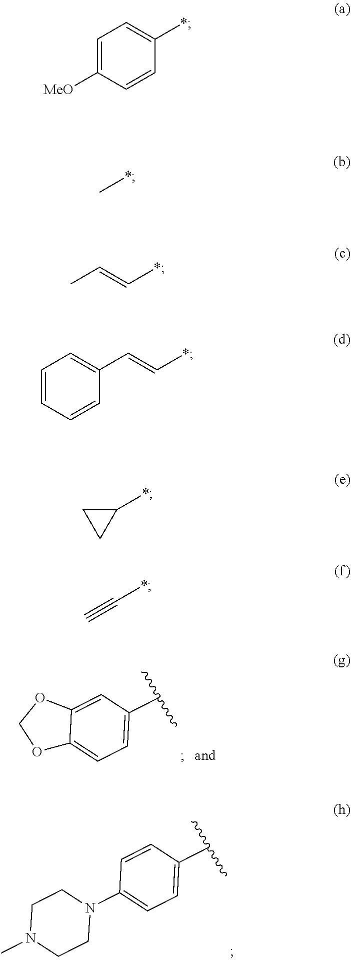
R^{1a} is selected from methyl and benzyl;
R^{LL1}, R^{LL2} and R^{11a} are as defined in claim 1.

123. The conjugate according to claim 114 wherein the modified antibody having at least one free conjugation site on each heavy chain is an IgG1, IgG2, IgG3 or IgG4 antibody.

124. The conjugate according to claim 123 wherein the modified antibody having at least one free conjugation site on each heavy chain is a human antibody or a humanized antibody.

125. The conjugate according to claim 123, wherein:
a) the native interchain cysteine residues have been substituted for amino acid residues lacking thiol groups; and/or
b) at least one additional substitutions in each heavy chain of an amino acid residue comprising a reactive group suitable for conjugation to a linker optionally wherein the additionally substituted amino acid is a cysteine or a non-natural amino acid.

126. A conjugate of formula II:
Ab′-(D^{L})p (II),
where D^{L} is of formula (III) 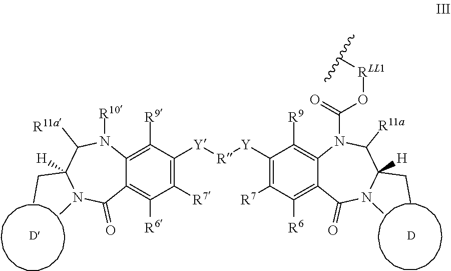
wherein D, R⁶, R⁷, R⁹, R^{11a}, Y, R″, Y′, D′, R^{6′}, R^{7′}, R^{9′}, R^{11a′} and R^{LL1} (including the presence or absence of double bonds between C2 and C3 and C2′ and C3′ respectively) are as defined in claim 1;
Ab′ is an antibody;
either:
(a) R^{10′} is H, and R^{11a′} is OH or OR^{A}, where R^{A} is C₁₋₄ alkyl;
(b) R^{10′} and R^{11a′} form a nitrogen-carbon double bond between the nitrogen and carbon atoms to which they are bound; or
(c) R^{10′} is H and R^{11a′} is SO_{z}M, where z is 2 or 3 and M is a monovalent pharmaceutically acceptable cation;
p is an integer of from 1 to 20.

127. The conjugate according to claim 126, wherein:
a) R^{10′} is H, and R^{11a′} is OH or OR^{A}, where R^{A} is C₁₋₄ alkyl or b) R^{10′} and R^{11a′} form a nitrogen-carbon double bond between the nitrogen and carbon atoms to which they are bound; or
c) R^{10′} is H and R^{11a′} is SO_{z}M, where z is 2 or 3 and M is a monovalent pharmaceutically acceptable cation.

128. The conjugate according to claim 126, wherein D^{L} is of formula IIIa, IIIb or IIIc: 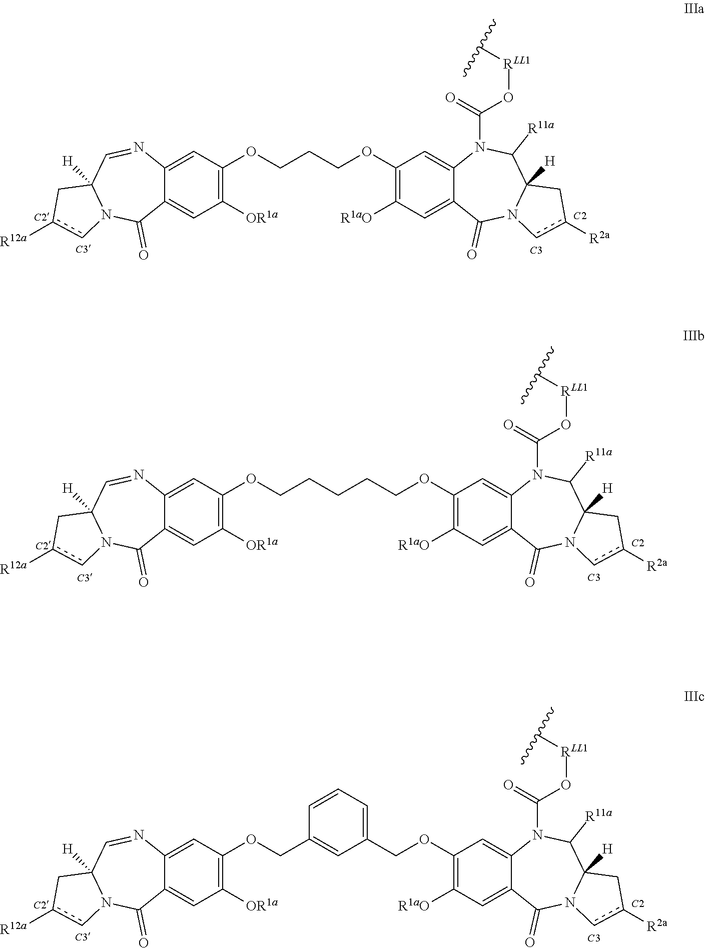
where R^{2a} and R^{22a} are the same and are selected from: 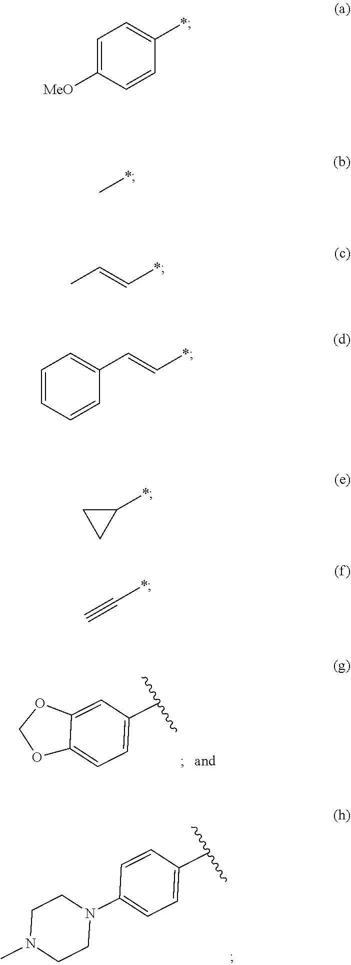
R^{1a} is selected from methyl and benzyl;
R^{LL1} is as defined in claim 1.

129. The conjugate according to claim 126, wherein p is an integer from 1 to 8.

130. The conjugate according to claim 114, wherein G^{LL} comprises a group selected from:

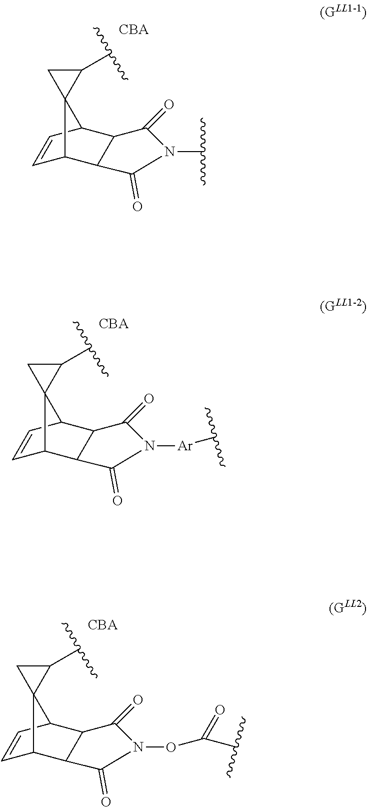

131. The conjugate according to claim 130, wherein G^{LL1-1}, G^{LL-2} or G^{LL2} is connected directly to X.

132. The conjugate according to claim 130, wherein G^{LL1-1}, G^{LL1-2} or G^{LL2} is connected to CBA via a group of formula IV: 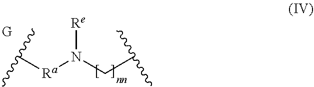
where G indicates where the group is connected to G^{LL1-1}, G^{LL1-2} and G^{LL2};
nn is from 1 to 4;
R^{a} represents a saturated or unsaturated branched or unbranched C₁₋₆ alkylene chain, wherein at least one carbon is replaced by a heteroatom selected from O, N, S(O)₀₋₃, wherein said chain is optionally, substituted by one or more groups independently selected from oxo, halogen, amino; and
R^{e} represents H, saturated or unsaturated branched or unbranched C₁₋₈ alkylene chain, wherein one or more carbons are optionally replaced by —O— and the chain is optionally substituted by one or more halogen atoms, N₃ or —C₂₋₅ alkynyl.

133. The conjugate according to claim 132, wherein:
a) R^{a} is selected from the group consisting of —(CH₂)ₘC(O)—, —CH₂(CH₃)C(O)—, —(CH₂)ₘCH₂OC(O)—,
—CHCHCH₂OC(O)—, and —OCH₂CH₂COC(O)— and m represents 0 or 1; and/or
b) R^{e} represents H or —CH₂OCH₂CH₂N₃.

134. The conjugate according to claim 132, wherein the group is incorporated in the antibody by the use of an unnatural amino acid of formula AA: 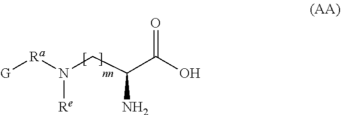
where G is a selected from a precursor of G^{LL1-1}, G^{LL1-2} and G^{LL2} optionally, wherein the unnatural amino acid is:

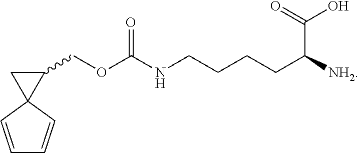 [SMILES: N[C@@H](CCCCNC(=O)OCC1CC12C=CC=C2)C(=O)O]

135. The conjugate according to claim 132, wherein the group is incorporated by conjugating a group of formula (BB) with the antibody: 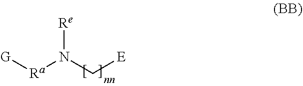
where E is a group —C(O)OR⁵⁵, R^{55′}, —NC(O)R⁶⁶, —C₂₋₅ alkylene, CH₂—O—NH₂ or halogen such as iodo;
R⁵⁵ represents C₁₋₆ alkyl, succinimide, C₆F₄H (tetrafluorohexyl), or H:
R^{55′} represents a sulfur bridging group, for example a dibromomaleimide, a dichloroacetone or a derivative of any one of the same,
R⁶⁶ represents: 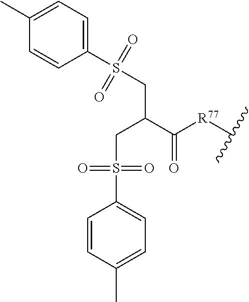
wherein
R⁷⁷ is C₁₋₆ alkylene optionally bearing one or more (such as one, two or three) groups selected from hydroxyl, sulfo, amino and —(OCH₂)_{V}C₂₋₆alkylene, and phenyl optionally bearing one or more (such as one, two or three) groups selected from hydroxyl, sulfo, amino and —(OCH₂)_{V}C₂₋₆alkylene,
v is an integer 1, 2, 3, 4 or 5 [symbol]
represents where the fragment is connected to the rest of the molecule, optionally wherein the compound of formula BB is:

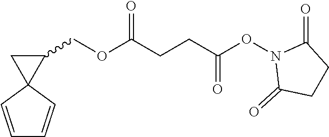 [SMILES: O=C(CCC(=O)ON1C(=O)CCC1=O)OCC1CC12C=CC=C2]

136. The conjugate according to claim 114, wherein Q^{X} is
a) an amino acid residue selected from Phe, Lys, Val, Ala, Cit, Leu, Ile, Arg, and Trp; or
b) a dipeptide residue selected from:
^{CO}-Phe-Lys-^{NH},
^{CO}-Val-Ala-^{NH},
^{CO}-Val-Lys-^{NH},
^{CO}-Ala-Lys-^{NH},
^{CO}-Val-Cit-^{NH},
^{CO}-Phe-Cit-^{NH},
^{CO}-Leu-Cit-^{NH},
^{CO}-Ile-Cit-^{NH},
^{CO}-Phe-Arg-^{NH}, and
^{CO}-Trp-Cit-^{NH}; or
c) a tripeptide residue.

137. The conjugate according to claim 114, wherein:
a) a is 0 to 3; and/or
b) b is 0 to 12; and/or
c) d is 0 to 3.

138. The conjugate according to claim 114, wherein a is 0, c is 1 and d is 2, and b is from 0 to 8.

139. The conjugate according to claim 114 of formula Id: 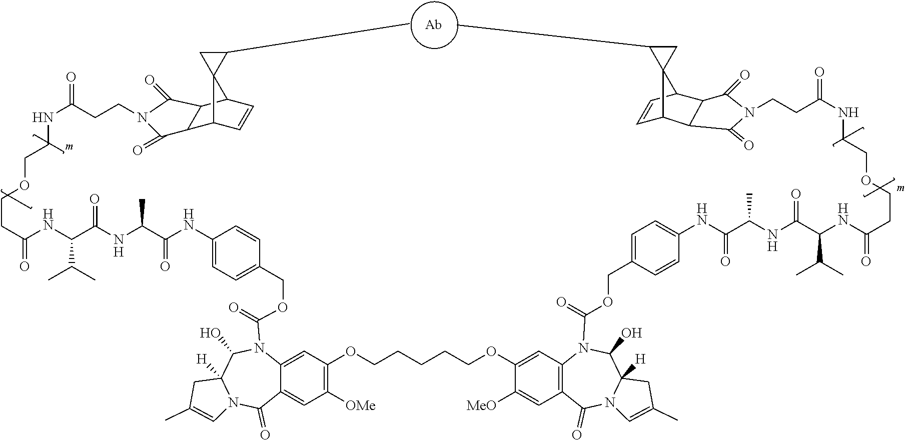
where m is an integer from 2 to 8.

140. The conjugate according to claim 126, wherein D^{L} is of formula IIId: 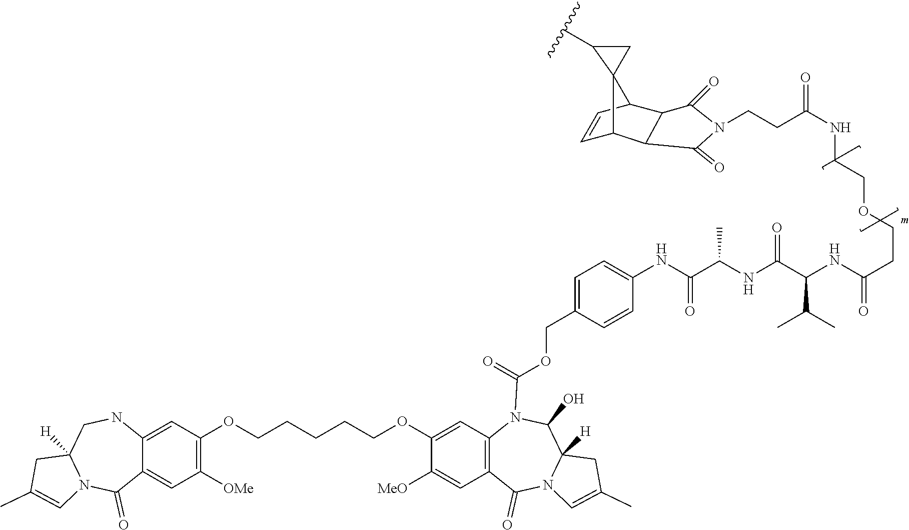
where m is an integer from 2 to 8.

141. A pharmaceutical composition comprising the conjugate of claim 114 and a pharmaceutically acceptable diluent, carrier or excipient.

142. A method of medical treatment comprising administering to a patient the pharmaceutical composition of claim 141.

143. The method of claim 142, wherein the method of medical treatment is for treating cancer.

144. The method of claim 143, wherein the patient is administered a chemotherapeutic agent, in combination with the conjugate.

145. A method of treating a mammal having a proliferative disease, comprising administering an effective amount of a conjugate according to claim 114.
